(19) United States
(12) Patent Application Publication
(10) Pub. No.: US 2025/0120985 A1
(43) Pub. Date: Apr. 17, 2025

(54) METHOD OF TREATING DIABETIC MACULAR EDEMA

(71) Applicant: Oculis Operations Sàrl, Lausanne (CH)

(72) Inventors: Riad Sherif, Lausanne (CH); Sabri Markabi, Hallandale Beach, FL (US); Pravin Dugel, Phoenix, AZ (US)

(73) Assignee: Oculis Operations Sàrl, Lausanne (CH)

(21) Appl. No.: 18/917,928

(22) Filed: Oct. 16, 2024

Related U.S. Application Data
(60) Provisional application No. 63/544,325, filed on Oct. 16, 2023.

(30) Foreign Application Priority Data
Oct. 16, 2023 (WO) PCT/EP2023/078697

(51) Int. Cl. A61K 31/573; A61K 9/00; A61K 47/10; A61K 47/18; A61K 47/69; A61P 27/02

(52) U.S. Cl. CPC A61K 31/573; A61K 9/0048; A61K 47/10; A61K 47/183; A61K 47/6951; A61P 27/02

(57) ABSTRACT

The present disclosure relates to a method of treating diabetic macular edema in a human subject in need thereof, said method comprising
(i) an induction phase of topically administering to an affected eye of said subject, an eye drop formulation comprising 1.5% (w/v) dexamethasone, at a dosing of 5 to 7 drops a day for 3, 4, 5, 6 weeks, followed by,
(ii) a maintenance phase of topically administering to an affected eye of said subject, said eye drop formulation comprising 1.5% (w/v) dexamethasone at a daily dosing less frequent than the induction phase for example 1-3 drops.

### CROSS-REFERENCE TO RELATED APPLICATIONS

This application claims the benefit of U.S. Provisional Application No. 63/544,325, filed Oct. 16, 2023, and International Patent Application No. PCT/EP2023/078697, filed in the European Receiving Office on Oct. 16, 2023. The entirety of each of these applications is incorporated herein for all purposes.

### TECHNICAL FIELD

The present disclosure describes an improved method of treating diabetic macular edema with topical drops in a human subject in need thereof, in a manner that provides a therapeutic effect that is similar to or better than the current state of the art using an ocular implant.

### BACKGROUND

Diabetic macular edema (DME) is the leading cause of visual loss and legal blindness in patients with diabetes. Currently, it is estimated to affect around 37 million people worldwide and, with the rise of diabetes, the prevalence is expected to increase to 53 million by 2040 (Yau et al. Diabetes Care 2012 March; 35 (3): 556-564; International Diabetes Federation-diabetesatlas.org Estimated diabetes prevalence worldwide in 2021: 537m, reaching 783m in 2045). DME is an irreversible and progressive complication of diabetic retinopathy and is related to consistently high blood sugar levels that damage nerves and blood vessels in the macula, the area of the retina responsible for sharp vision. DME occurs when blood vessels in the retina swell, and then leak, leading to a fluid build-up (edema) into the retina. There remains a significant need for safer, more effective, longer lasting, and less burdensome treatments for DME patients.

Most ocular conditions can be treated and/or managed to reduce negative effects, including total blindness. However, current treatments for ocular conditions are limited by the difficulty in delivering effective doses of drugs to target tissues in the eye. In current treatments, topical administration of eye drops is the preferred means of drug administration to the eye due to the convenience and safety of eye drops in comparison to other routes of ophthalmic drug administration such as intravitreal injections and implants (Le Souriais, C., et al., 1998. Progress in Retinal and Eye Research 17, 33-58). Drugs are mainly transported by passive diffusion from the eye surface into the eye and surrounding tissues where, according to Fick's law, the drug is driven into the eye by the gradient of dissolved drug molecules. The passive drug diffusion into the eye is hampered by three major obstacles (Gan, L., et al, 2013. Drug Discov. Today 18, 290-297; Loftsson, T., et al., 2008. Pharmazie 63, 171-179; Urtti, A, 2006. Adv. Drug Del. Rev. 58, 1131-1135).

Achieving therapeutic concentrations of drugs in the posterior segment of the eye following topical application using eye drops, however, is highly challenging. Currently more invasive methods of application such as intravitreal injection, vitreous implants, periocular injection, or systemic dosing are the only options available. Such invasive methods have numerous, respective disadvantages.

For example, dexamethasone as an intravitreal implant is registered for use for DME, macular edema in retinal vein occlusions, and posterior uveitis (OZURDEX® 700 micrograms intravitreal implant in applicator Full US Prescribing Information, available at https://www.ozurdex.com/). In 2 separate DME clinical studies of OZURDEX®, the mean change baseline in best corrected visual acuity (BCVA) letters at 3-months for all patients (pooled) was 6. At 39-months from baseline, the mean change for all patients (pooled) in BCVA letters was 2.2. The percentage of all study subjects (pooled) experiencing a ≥15 letter improvement at 3-months from baseline BCVA was 13%, and 20% at 39-months. The use of OZURDEX®, however, is associated with significant ocular adverse events. As noted in the US Prescribing Information, intravitreal implants are associated with endophthalmitis, eye inflammation, increased intraocular pressure, and retinal detachments. Separately, as noted in the US Prescribing Information, the use of corticosteroids may produce posterior subcapsular cataracts, increased intraocular pressure, glaucoma, and may enhance the establishment of secondary ocular infections due to bacteria, fungi, or viruses. The US Prescribing Information for OZURDEX® reports that 68% of study patients receiving OZURDEX® experienced cataracts, which includes cataract, cataract nuclear, cataract subcapsular, and lenticular opacities, and 61% of the study patients that developed cataracts underwent surgery. In addition, 28% of study subjects receiving OZURDEX® experienced an IOP elevation of ≥10 mm Hg from baseline at any visit, with 15% of study subjects experiencing an IOP of ≥30 mm Hg at any visit, and 6% of study subject experiencing an IOP of ≥35 mm Hg at any visit.

Although current eye drop formulations are the preferred route for the treatment of anterior segment conditions, drug penetration, particularly of insoluble molecular entities, is often limited due to pre-corneal losses in tear fluid and nasolacrimal drainage (Johannsdottir S, Jansook P, Stefansson E., Topical drug delivery to the posterior segment of the eye: Dexamethasone concentrations in various eye tissues after topical administration for up to 15 days to rabbits. J Drug Deliv Sci Tech. 2018; 45:449-54). Dexamethasone, dosed as commercially-available eye drops, is used to treat a number of indications including inflammatory conditions of the conjunctiva, cornea, and other tissues of the anterior segment of the eye, such as anterior uveitis, iritis, cyclitis, allergic and vernal conjunctivitis, herpes zoster keratitis, superficial punctate keratitis, and non-specific superficial keratitis (MAXIDEX® 0.1%, w/v, eye drops, US Prescribing Information (Alcon Laboratories Inc, Fort Worth, Texas, USA. MAXIDEX®-dexamethasone ophthalmic suspension. Full Prescribing Information. Available at: https://www.accessdata.fda.gov/drugsatfda_docs/label/2017/013422s0451bl.pdf. Accessed Nov. 12 2020).

In order to increase concentrations of topically-administered drugs in the posterior segment of the eye, an ophthalmic suspension using γ-CD has been developed (Jansook P, Stéfansson E, Thorsteinsdóttir M, Sigurdsson B B, Kristjánsdóttir SS, Bas J F,et al. Cyclodextrin solubilization of carbonic anhydrase inhibitor drugs: formulation of dorzolamide eye drop microparticle suspension. Eur J Pharm Biopharm. 2010; 76:208-14).

Recently, applicants have described preparation and testing of cyclodextrin-based eye drops containing dexamethasone 1.5% w/v as the active ingredient. Such cyclodextrin-based eye drops containing dexamethasone are described in particular in WO2007/12974, WO2018/100434 and WO2021/001366.

Tanito et al 2011 (Opthalmol Vis Sci 2011; 52:7944-8) reported a small, non-controlled pilot study (19 patients) with a topical 1.5% (w/v) dexamethasone-cyclodextrin eye drop formulation in DME patients. The subjects were treated with a treatment regimen of either 3 times a day (N=11) or 6 times a day (N=8) for 4 weeks. The study showed a central macular thickness (CMT) decrease of at least 10% in 63% of patients treated, with mean change CMT reduction across the study population of about 20% at week 4 compared to baseline. Tanito et al. report, however, that the change in CMT was not significantly different between the patients in the 3 times a day and 6 times a day treatment regimens. In 74% of subjects, visual acuity measured by the logarithm of the minimum angle of resolution (log MAR) had improved ≥0.1 at week 4 (equivalent to 5 BVCA letters), with a mean improvement at week 4 of 0.15 log MAR from baseline (equivalent to 7.5 BCVA letters). Following cessation of treatment at week 4, however, the CMT and visual acuity (log MAR) of these patients at week 8 was not significantly different than that at baseline. Given the reversion back to baseline, the authors conclude that the 4-week treatment did not “provide a sustained treatment effect.” Importantly, Tanito et al. note that a mean increase in interocular pressure (IOP) of 2 mm in treated subjects was noted at week 4, and that corticosteroid use in the eye is associated with increased IOP which can be expected in longer studies, the development of cataracts, and infections.

Ohira et al 2015 (Acta Ophthalmol. 2015: 93: 610-15) also reported a small prospective, randomized, controlled trial using a topical 1.5% dexamethasone-cyclodextrin eye drop formulation in DME patients (N=12), with a dosing regimen of three drops a day for 4 weeks, two drops a day for 4 weeks, and 1 drop a day for 4 weeks. A mean log MAR improvement of 0.09+/−0.15 at 4 weeks (equivalent to 4.5 BCVA letters), 0.11+/−0.13 at 8 weeks (equivalent to 5.5 BVCA letters), and 0.09+/−0.17 at 12 weeks (equivalent to 4.5 BCVA letters) was seen in patients receiving the 1.5% dexamethasone-cyclodextrin eye drop formulation compared to baseline. Four patients achieved a log MAR improvement of ≥0.3 (equivalent to ≥15 BVCA letters). The mean improvement in log MAR at week 16, however, had fallen to 0.01+/−0.26 (equivalent to 0.5 BVCA letters), which was statistically insignificant compared to baseline. A reduction of mean CMT of 99+/−169 at 4-weeks, 141+/−211 at 8 weeks, and 76+/−139 at 12 weeks was seen in subjects receiving the 1.5% dexamethasone-cyclodextrin eye drop formulation compared to baseline. Like the visual acuity measurements, however, the mean CMT measurements also were not statistically significant at 4 weeks post-treatment (week 16). Ohira et al. note that IOP in the 1.5% dexamethasone-cyclodextrin eye-drop receiving subjects was significantly higher at follow-up visits compared to baseline, with 3 subjects experiencing an IOP of greater than 30 mm requiring pressure-lowering medications. Similar to Tanito et al., Ohira et al. note that the use of corticosteroids in the eye has a well-known side-effect profile, and that IOP rose significantly in the subjects receiving 1.5% dexamethasone-cyclodextrin drops. In addition, Ohira et al. note that while “cataract formation was not noted, [it] would be expected with longer duration of treatment.”

Stefansson et al reported the results of a phase II study on topical treatment of DME with a dosing regimen of 3 drops a day (N=99) of a 1.5% (w/v) dexamethasone-cyclodextrin formulation for 12 weeks (Acta Ophthalmologica 2023, 101:22-33). At week 12, mean CMT in the population was −53.6 μM, with mean change in BCVA letter score from baseline at week 12 of +2.6 letters. Five percent of subjects experienced a gain of ≥15 letters at 12 weeks. A mean IOP pressure increase of 1.17 mmHg at week 2 to 4.53 mmHg at week 12 was noted in subjects receiving the 1.5% dexamethasone-cyclodextrin formulation, with 14% of patients having an IOP increase of ≥10 mmHg at week 12, and 7 patients receiving IOP-reducing medication. Stefansson et al. note that mean CMT measurements “began to return towards baseline over the 4 week follow-up period.”

There is still a need to provide long-term suitable treatment alternatives to patients for the treatment of DME.

### SUMMARY

The inventors have surprisingly found that administering an induction phase of topical 1.5% (w/v) dexamethasone-cyclodextrin eye drop formulation at a dosing of 5-7 drops a day, for example, 6 drops a day for 4-6 weeks, for example, 6 weeks followed by a maintenance phase at a daily dosing of 1-3 drops a day, for example, 2 or 3 drops a day provides extended improved visual acuity and reduced central macular thickness (CMT) which is similar to or exceeds currently approved dexamethasone intravitreal implant OZURDEX® and previous topical administration regimes. This new dosage regimen surprisingly achieves at least similar results as the intravitreal implant, but with a more convenient and less invasive topical administration. The inventors have found that the use of a high induction dose, for example, 6 drops a day of a 1.5% (w/v) dexamethasone-cyclodextrin eye drop formulation for 6 weeks, provides for a rapidly induced therapeutic response resulting in significant visual acuity improvements which surprisingly are similar to or exceed those seen with OZURDEX® during the same period, and which can be maintained long-term with a lower maintenance dose. This increased induction dose may be particularly important for quickly inducing a therapeutic response in those DME patients who may not be prone to response at a lower dose. Thus, the ability to achieve significant and lasting visual acuity and CMT improvements using a topically administered eye drop to treat a disorder in the back of the eye represents a significant and important advancement in the treatment of DME, from both a therapeutic efficacy and patient compliance perspective. Importantly, the extended gains in visual acuity and reduced CMT are achieved without a significant increase in detrimental dexamethasone-associated ocular side effects compared to the prior art such as increased ocular pressure (IOP), cataracts, and/or the development of ocular infections.

As described further herein, it has been established through extensive human clinical trial testing that the use of an increased induction dose of a topically administered 1.5% (w/v) dexamethasone-cyclodextrin eye drop formulation during an induction phase for 6 weeks, followed by an extended maintenance dose provides for significantly improved and sustained visual acuity in patients suffering from DME. Human clinical trials described further in Example 2 have shown that the use of an induction dose and maintenance dose as described herein provided a mean improvement in visual acuity at week 6 of 7.2 BCVA letters, at week 8 of 7.1 BCVA letters, and at week 12 of 7.6 BCVA letters compared to baseline (see FIG. 1), with greater than 27% of patients experiencing an increase of ≥15 letters at 12-weeks (see Example 2, Table 1). Comparatively, pooled study patients receiving the intravitreal implant OZURDEX® were shown to have a mean improvement in visual acuity at week 12 of 6 letters, with only 13% of pooled study patients experiencing an increase of ≥15 letters at 12 weeks (see OZURDEX® US Prescribing Information; Table 4).

Likewise, Ohira et al. and Stefansson et al. showed significantly less improvement in mean improvement in BCVA letters compared to the induction dose/maintenance dose described herein. For example, Ohira et al., report a mean improvement in BCVA letters of 5.5 from baseline at week 8 and 4.5 letters from baseline at week 12, while Stefansson et al. report a mean improvement of only 2.6 letters from baseline at week 12.

The use of an increased induction dose followed by an extended maintenance dose as described herein also showed an extended reduction in mean central subfield thickness (CST) in the study patient population, indicating that the maintenance dose sufficiently maintains the reduction in CST induced by the increased induction dose (see Example 2, FIG. 2).

Notably, the therapeutic improvements achieved using the increased induction dose-maintenance dose regime described herein are not associated with a significant increase in ocular adverse events associated with the use of dexamethasone compared to prior art dexamethasone dosing regimes (see Example 2, Table 2, Table 3). For example, despite the significant increase in the dose administered during the induction phase, only 16.5% of subjects experienced an IOP increase of ≥10 mm Hg (see Example 2, Table 2) at any visit during the initial 12-weeks of treatment, which is similar to the 14% of subjects experiencing an IOP increase of ≥10 mm Hg using the dosing regime described in Ohira et al. Comparatively, 28% of patients receiving OZURDEX® experienced an IOP increase of ≥10 mm Hg at any event across the reported 39-month clinical trial studies (see OZURDEX® US Prescribing Information). As shown in FIG. 3, the mean change in IOP from baseline in the treated study patients using the dosing regimen described herein across the initial 12-week study ranged was 0.64 mm Hg at week 2 to 2.79 mm Hg at week 12.

Likewise, initial data on cataract development in study subjects receiving the increased induction dose-maintenance dose administration regimen described herein indicates that cataract development was not significantly different or accelerated compared to the control group (1.0% v. 2.1%, respectively). This initial data of non-acceleration compared to control supports the use of the induction phase-maintenance phase dosing regimen described herein to achieve therapeutic efficacy in controlling DME and improving visual acuity with at least a similar, if not better, safety profile compared to OZURDEX®, wherein 68% of study subjects receiving OZURDEX® developed cataracts compared to 21% of control subjects during the 39-month OZURDEX® study period (see OZURDEX® US Prescribing Information).

Accordingly, a first object of the present disclosure is a method of treating diabetic macular edema (DME) in a human subject in need thereof, said method comprising
(i) an induction phase of topically administering to an affected eye of said subject, an eye drop formulation comprising 1.5% (w/v) dexamethasone, at a dosing of 5 to 7 drops a day, followed by,
(ii) a maintenance phase of topically administering to an affected eye of said subject, said eye drop formulation comprising 1.5% (w/v) dexamethasone at a daily dosing less frequent than the induction phase.

The disclosure also relates to an eye drop formulation comprising 1.5% (w/v) dexamethasone for use in a method for treating diabetic macular edema in a subject in need thereof, said method comprising
(i) an induction phase of topically administering to an affected eye of said subject, an eye drop formulation comprising 1.5% (w/v) dexamethasone, at a dosing of 5 to 7 drops a day, followed by,
(ii) a maintenance phase of topically administering to an affected eye of said subject, said eye drop formulation comprising 1.5% (w/v) dexamethasone at a daily dosing less frequent than the induction phase.

The disclosure also relates to the use of dexamethasone in the manufacture of an eye drop formulation comprising 1.5% (w/v) dexamethasone for treating diabetic macular edema in a subject in need thereof, said treatment comprising
(i) an induction phase of topically administering to an affected eye of said subject, an eye drop formulation comprising 1.5% (w/v) dexamethasone, at a dosing of 5 to 7 drops a day, followed by,
(ii) a maintenance phase of topically administering to an affected eye of said subject, said eye drop formulation comprising 1.5% (w/v) dexamethasone at a daily dosing less frequent than the induction phase.

In some embodiments, the induction phase is administered for at least 5 days to about 4 to 6 weeks. In some embodiments, the induction phase is administered for 4 to 6 weeks. In some embodiments, the induction phase is administered for 6 weeks. In some embodiments, the induction phase is administered as 5 drops a day. In some embodiments, the induction phase is administered as 6 drops a day. In some embodiments, the induction phase is administered as 7 drops a day. In some embodiments, the induction phase is administered as 6 drops a day for 6 weeks. In some embodiments, the maintenance phase is administered as 1 drop a day. In some embodiments, the maintenance phase is administered as 2 drops a day. In some embodiments, the maintenance phase is administered as 3 drops a day. In some embodiments, the eye drop formulation is administered during the maintenance phase for at least 6 weeks, 7 weeks, 8 weeks, 9 weeks, 10 weeks, 11 weeks, 12 weeks, 18 weeks, 24 weeks, 30 weeks, 36 weeks, 40 weeks, 46 weeks, or greater than 46 weeks, 18 months, 24 months, or longer, including indefinitely.

In some embodiments, the administration regime described herein results in an improved BCVA/ETDRS letter assessment across a population of treated patients of ≥15 letters gained at 12-months from baseline of at least 25%, at least 30%, at least 35%, or greater than 35%.

In specific embodiments, said eye drop formulation comprises or consists essentially of:
−1.5% of dexamethasone,
12 to 16% of γ-cyclodextrin;
−2.2 to 2.7% of polymer, typically poloxamer;
−0 to 0.1% of chelating agent, for example 0.1% of chelating agent, for example, but not limited to, disodium edetate;
−0 to 0.6% of an additive to prevent the oxidation of the dexamethasone, for example between 0.05% and 0.5%, or between 0.05% and 0.3%, of an additive to prevent the oxidation of the corticosteroid, for example, but not limited to, phenolic antioxidants or reducing agents, such as water-soluble natural antioxidants, sodium thiosulfate, L-methionine, or 3,4-dihydroxybenzoic acid;
−0 to 1% of electrolyte, for example 0.57% of electrolyte, for example, but not limited to, sodium chloride; and
water;
wherein the % are % by weight based on the volume of the composition. In some embodiments, the poloxamer is poloxamer 407. In some embodiment, the eye drop formulation includes non-derivatized, natural γ-cyclodextrin.

In preferred embodiments, said eye drop formulation comprises or consists essentially of:
−1.5% of dexamethasone;
−14% of γ-cyclodextrin;
−2.5% of poloxamer;
−0 to 0.1% of chelating agent, for example 0.1% of disodium edetate;
−0 to 1% of electrolyte, for example 0.57% of sodium chloride;
−0% to 0.6% of an additive to prevent the oxidation of the dexamethasone, for example. 0.3% STS; and
water;
wherein the % are % by weight based on the volume of the composition. In some embodiments, the poloxamer is poloxamer 407. In some embodiments, the eye drop formulation includes non-derivatized, natural γ-cyclodextrin.

Advantageously, the eye-drop formulations described herein are particularly useful in treating DME, as they are formulated to comprise a microsuspension comprising solid complexes of dexamethasone and γ-cyclodextrin comprising, for example, a diameter D50 ranging from about 1 μm to 20 μm. These microparticles provide extended release of dexamethasone into the eye, allowing sufficient concentrations of the dexamethasone to reach the posterior of the eye, thus effectively treating DME. In some embodiments, the microsuspension comprises microparticles with a diameter of D₅₀ of between about 1 μM and 10 μM. In some embodiments, the microsuspension has a viscosity (cP) of between about 2 and 16 cP. In some embodiments, the eye-drop formulation has a pH of between about 4.5 and 7. In some embodiments, the eye-drop formulation has a pH of between about 5 and 6. In specific embodiments, said eye drop formulation is preservative-free.

### BRIEF DESCRIPTION OF THE DRAWINGS

FIG. 1: Study DX219, Stage 1, Mean Change from Baseline in BCVA (Best corrected visual acuity) ETDRS better Score at Each Follow-up Visit-Full Study (ITT). BCVA, best corrected visual acuity; ITT, intention-to-treat; SE, standard error.

FIG. 2: Study 219, Stage 1, Change in CST Compared With Baseline-Full Study (ITT). CST, central subfield thickness; LS, least squares; SE, standard error. Imputation rules are applied based on a pattern-mixture model approach.

FIG. 3: Study 219, Stage 1, minimal mean intraocular pressure (IOP) changes are observed across the induction and maintenance phases. Mean changes in IOP from baseline, measured in millimeter of mercury (mm Hg), with standard deviation (SD) represented in brackets are illustrated on the y-axis for different time points during treatment measured as weeks from start of treatment as represented on the x-axis for either OCS-01 (dark gray bars) or vehicle (light gray bars). Mean (SD) baseline IOP: OCS-01, 15.3 (3.1) mm Hg; vehicle, 14.7 (3.0) mm Hg.

### DETAILED DESCRIPTION

### Definitions

As used herein, the term “treating” includes reversing, alleviating, or inhibiting the progression of a disease, disorder, or condition to which such term applies, or one or more symptoms or manifestations of such disease, disorder or condition.

In certain aspects the present invention can be used to treat a disorder, for example DME. In other aspects the present invention can be used for preventing a disorder, for example DME. Preventing refers to causing a disease, disorder, condition, or symptom or manifestation of such, or worsening of the severity of such, not to occur. Accordingly, the presently disclosed compositions can also be administered prophylactically to prevent or reduce the incidence or recurrence of the disease, disorder, or condition.

As used herein the term “% by weight of a compound X based on the volume of the composition”, also abbreviated as “% (w/v)”, corresponds to the amount of compound X in grams that is introduced in 100 mL of the composition.

As used herein the term “microparticle” refers to a particle having a diameter D50 of about 1 μm to about 100 μm. In exemplary embodiments, the diameter, which can be D50, is 1 μm or greater to about 100 μm, 1 μm to 50 μm, 1 μm to 40 μm, 1 μm to 35 μm, 1 μm to 30 μm, 1 μm to 25 μm, 1 μm to 20 μm, 1 μm to 15 μm, 1 μm to 10 μm, or 1 μm to 5 μm.

The term “microsuspension” is intended to mean a composition, typically an ophthalmic preparation, comprising microparticles suspended in a liquid phase.

As used herein, the term “eye drop formulation” refers to a pharmaceutical composition comprising an active therapeutic agent (drug) in a medium suitable for topical application of said drug to the eye of a subject in need thereof.

The term “ophthalmically acceptable medium” is intended to mean a medium suitable for ophthalmic administration of the composition. The ophthalmically acceptable medium is preferably a liquid, for example water.

The term “about” refers to +/−10% of the recited value. For example, about 0.3% is between 0.27% and 0.33%.

### Eye Drop Formulations

### Dexamethasone as the Active Therapeutic Agent

The eye drop formulation for use according to the present disclosure comprises dexamethasone, a betamethasone-type corticosteroid which is a glucocorticoid having a C₁₆ methyl substitution, or its pharmaceutically acceptable salts, esters or derivatives thereof.

Dexamethasone is commercially available and is the following formula:

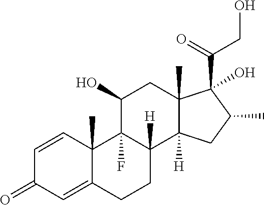

The concentration of the dexamethasone in the eye drop formulation for use according to the present disclosure may be from about 10 mg/mL to about 25 mg/mL.
In specific embodiments, the amount of dexamethasone in the eye drop formulation is between about 1.0-2.5%, for example, 1.5%, by weight of drug based on the volume of the formulation (w/v).

In specific embodiments, the eye drop formulation comprises γ-cyclodextrin.

Cyclodextrins are cyclic oligosaccharides containing 6 (α-cyclodextrin), 7 (β-cyclodextrin), and 8 (γ-cyclodextrin) glucopyranose monomers linked via α-1,4-glycoside bonds. α-Cyclodextrin, β-cyclodextrin and γ-cyclodextrin are natural products formed by microbial degradation of starch. The outer surface of the doughnut shaped cyclodextrin molecules is hydrophilic, bearing numerous hydroxyl groups, but their central cavity is somewhat lipophilic (Kurkov, S. V., Loftsson, T., 2013. Int J Pharm 453, 167-180; Loftsson, T., Brewster, M. E., 1996. Journal of Pharmaceutical Sciences 85, 1017-1025). In addition to the three natural cyclodextrins, numerous water-soluble cyclodextrin derivatives have been synthesized and tested as drug carriers, including cyclodextrin polymers (Stella, V. J., He, Q., 2008. Cyclodextrins. Tox. Pathol. 36, 30-42).

Cyclodextrins can enhance the solubility and bioavailability of hydrophobic compounds. In aqueous solutions, cyclodextrins form inclusion complexes with many drugs by taking up a drug molecule, or more frequently some lipophilic moiety of the molecule, into the central cavity. This property has been used for drug formulation and drug delivery purposes. Formation of drug/cyclodextrin inclusion complexes, their effect on the physicochemical properties of drugs, their effect on the ability of drugs to permeate biomembranes and the usage of cyclodextrins in pharmaceutical products have been reviewed (Loftsson, T., Brewster, M. E., 2010. Journal of Pharmacy and Pharmacology 62, 1607-1621; Loftsson, T., Brewster, M. E., 2011. J. Pharm. Pharmacol. 63, 1119-1135; Loftsson, T., Jarvinen, T., 1999. Advanced Drug Delivery Reviews 36, 59-79).

Certain drug/cyclodextrin complexes are able to self-assemble in aqueous solutions to form nano- and micro-sized aggregates and micellar-like structures that are also able to solubilize poorly soluble drugs through non-inclusion complexation and micellar-like solubilization (Messner, M., et al, 2010. Int J Pharm 387, 199-208). In general, the tendency of cyclodextrins to self-assemble and form aggregates increases upon formation of drug/cyclodextrin complexes and the aggregation increases with increasing concentration of drug/cyclodextrin complexes. In general, hydrophilic cyclodextrin derivatives, such as 2-hydroxypropyl-β-cyclodextrin and 2-hydroxypropyl-γ-cyclodextrin, and their complexes are freely soluble in water. On the other hand, the natural α-cyclodextrin, β-cyclodextrin and γ-cyclodextrin and their complexes have limited solubility in pure water or 129.5±0.7, 18.4±0.2 and 249.2±0.2 mg/mL, respectively, at 25° C. (Sabadini E., et al., 2006. Carbohydr Res 341, 270-274). Dexamethasone forms advantageous microparticle complexes with γ-cyclodextrin.

In specific embodiments, the eye drop formulation for use according to the present disclosure comprises at least γ-cyclodextrin. In some embodiments, γ-cyclodextrin is the only cyclodextrin in the eye drop formulation.

In specific embodiments, the eye drop formulation comprises γ-cyclodextrin or a combination of γ-cyclodextrin and hydrophilic cyclodextrin derivatives, such as 2-hydroxypropyl-β-cyclodextrin and 2-hydroxypropyl-γ-cyclodextrin.

In specific embodiments, the eye drop formulation for use according to the disclosure comprise solid complexes of a drug and a cyclodextrin. The complex comprising dexamethasone and a cyclodextrin may be referred to as a “dexamethasone/cyclodextrin complex”. When the cyclodextrin is γ-cyclodextrin, the complex comprising dexamethasone and γ-cyclodextrin may be referred to as a “dexamethasone/γ-cyclodextrin complex”.

In certain embodiments with 1.5% (w/v) dexamethasone as the drug, the amount of γ-cyclodextrin, in the eye drop formulation for use according to the present disclosure is from 5 to 25%, in particular from 12 to 16%, preferably about 14% by weight of γ-cyclodextrin based on the volume of the formulation.

The complexes of the eye drop formulation form aggregates, hereafter referred as complex aggregate. The complex aggregate may correspond to an aggregate of a plurality of complexes, in particular a plurality of inclusion complexes comprising dexamethasone and a γ-cyclodextrin.

According to one preferred embodiment, the eye drop formulation for use according to the present disclosure is a microsuspension comprising solid complexes of dexamethasone and γ-cyclodextrin. This does not exclude that the aggregate includes also γ-cyclodextrin not complexed with dexamethasone.

According to one more preferred embodiment, the eye drop formulation for use according to the present disclosure is a microsuspension comprising solid complexes of dexamethasone, γ-cyclodextrin and poloxamer. This does not exclude that the aggregate includes also cyclodextrin not complexed with dexamethasone and/or without poloxamer.

In specific embodiments, the eye drop formulation for use according to the present disclosure is a microsuspension comprising microparticles formed by the aggregates of a diameter D50 of less than about 90 μm, in particular about 1 μm to about 90 μm. In one embodiment, the diameter D50 of the particles of the microsuspension may be in the range of about 1 μm to about 25 μm, in particular about 1 μm to about 20 μm, more particularly about 1 μm to about 10 μm, even more particularly about 2 μm to about 10 μm.

The microparticles may comprise aggregates of cyclodextrins and/or dexamethasone/cyclodextrin/polymer complexes. In specific embodiments, the particles of the microsuspension comprise aggregates of γ-cyclodextrin and/or dexamethasone/γ-cyclodextrin/poloxamer complexes.

The diameter and/or size of a particle or complex can be measured according to any method known to those of ordinary skill in the art. For example, the diameter D₅₀ is measured by laser diffraction particle size analysis. Generally, there are a limited number of techniques for measuring/evaluating cyclodextrin/drug particle or complex diameter and/or size. In particular, persons of ordinary skill in this field know that the physical properties (e.g. particle size, diameter, average diameter, mean particle size, etc.) are typically evaluated/measured using such limited, typical known techniques. For example, such known techniques are described in Int. J. Pharm. 493 (2015), 86-95, which is incorporated by reference herein in its entirety. In addition, such limited, known measurement/evaluation techniques were known in the art as evidenced by other technical references such as, for example, European Pharmacopoeia (2.9.31 Particle size analysis by laser diffraction, January 2010), and Saurabh Bhatia, Nanoparticles types, classification, characterization, fabrication methods and drug delivery applications, Chapter 2, Natural Polymer Drug Delivery Systems, PP. 33-94, Springer, 2016, which are also incorporated by reference herein in their entireties.

In particular, 60 to 95% by weight, more particularly 70 to 90% by weight, of the drug in the composition may be in the form of a solid complex of drug and γ-cyclodextrin. Even more particularly, 5 to 40% by weight, in particular 10 to 30% by weight, of the drug in the composition may be in dissolved form. The dissolved form includes uncomplexed drug that is dissolved in the liquid phase and complexes of drug and cyclodextrin that are dissolved in the liquid phase as well as water-soluble nanoparticles consisting of drug/γ-cyclodextrin complex aggregates.

Preferably, 0% to 0.5% by weight of the drug in the composition may be in uncomplexed solid form. As such, the composition of the disclosure may be substantially free of solid uncomplexed particles of drug.

In one embodiment, the microsuspension may comprise about 70% to about 99% of the drug dexamethasone in microparticles and about 1% to about 30% of the drug dexamethasone in nanoparticles. More particularly, the microsuspension may comprise about 80% to about 95% of the drug dexamethasone in microparticles having a diameter of about 1 μm to about 90 μm, and about 20% to about 5% of the drug dexamethasone in nanoparticles. The microsuspension may comprise about 80% of the drug dexamethasone in microparticles having a diameter of about 1 μm to about 20 μm, and about 20% of the drug dexamethasone in nanoparticles.

In another embodiment, the microsuspension may comprise about 40% to about 99% of the drug dexamethasone in microparticles and about 1% to about 60% of the drug dexamethasone in nanoparticles or water-soluble dexamethasone/cyclodextrin complexes. In particular, the microsuspension may comprise about 80% to about 95% of the drug dexamethasone in microparticles having a diameter of about 1 μm to about 10 μm, and about 5% to about 20% of the drug dexamethasone in nanoparticles or water-soluble dexamethasone/γ-cyclodextrin complexes.

Examples of eye drop formulation comprising dexamethasone/cyclodextrin complexes and their manufacturing methods are disclosed in WO2018/100434 and US20230042785, which are hereby incorporated by reference. Additional examples of compositions comprising dexamethasone/cyclodextrin complexes are also disclosed in CN115837027A.

### Additive

According to a specific embodiment the eye drop formulation for use according to the present disclosure comprises water and optionally an additive selected from the group consisting of a preservative, a chelating agent, an electrolyte, and combinations thereof. In particular, the ophthalmically acceptable medium may comprise a preservative.

A preservative may be used to limit bacterial proliferation in the composition. Examples of preservatives are benzalkonium chloride, chlorobutanol, thimerosal, phenylmercuric acetate, phenylmercuric nitrate, methylparaben, phenylethyl alcohol, and combinations thereof. The amount of preservative in the eye drop formulation for use according to the disclosure may be 0 to 1%, in particular 0.001 to 0.5%, more particularly 0.005 to 0.1%, even more particularly 0.01 to 0.04%, by weight of preservative based on the volume of the composition. In a preferred embodiment, the eye drop formulation is preservative free.

In particular, the eye drop formulation may comprise a chelating agent. An example of a suitable chelating agent is disodium edetate. The amount of chelating agent in the composition of the disclosure may be 0 to 1%, in particular 0.01 to 0.5%, more particularly 0 to 0.2%, even more particularly 0.08 to 0.2%, for example of about 0.1% by weight of chelating agent based on the volume of the composition.

In particular, the eye drop formulation may comprise an electrolyte. An electrolyte may especially be used to make the composition isotonic. Examples of suitable electrolytes include sodium chloride, potassium chloride, and combinations thereof. Preferably, the electrolyte is sodium chloride. The amount of electrolyte in the composition of the disclosure may be 0 to 2%, in particular 0.1 to 1.5%, more particularly 0 to 1%, even more particularly 0.2 to 1%, for example of about 0.57% by weight of electrolyte based on the volume of the composition.

The eye drop formulation may further comprise a polymer. In particular, said polymer may be a water-soluble polymer. Moreover, said polymer may be a viscosity enhancing polymer. The term “viscosity enhancing polymer” is intended to mean a polymer that increases the viscosity of a liquid. The polymer increases the viscosity of the eye drop formulation for use according to the present disclosure. The increase of viscosity results is an enhanced physical stability of the composition. As such, the composition is less prone to sedimentation of the solid complex when it comprises a polymer. In specific embodiments, the polymer, for example poloxamer, is part of dexamethasone/g-cyclodextrin complexes of the microsuspension.

In specific embodiments, the polymer is selected so that the microsuspension has a viscosity comprised between 1 cP and 30 cP as measured according to European Pharmacopeia 2.2.10.

In specific embodiments, the polymer is used to disperse and stabilize the particles of the microsuspension, increase the viscosity and impart mucoadhesive characteristics.

The polymer may thus be considered as a polymeric stabilizing agent. In particular, the polymer may be a surface active polymer. The term “surface active polymer” is intended to mean a polymer that exhibits surfactant properties. Surface active polymers may, for example, comprise hydrophobic chains grafted to a hydrophilic backbone polymer; hydrophilic chains grafted to a hydrophobic backbone; or alternating hydrophilic and hydrophobic segments. The first two types are called graft copolymers and the third type is named block copolymer.

In one embodiment, the eye drop formulation for use according to the disclosure comprises a polymer selected from the group consisting of a polyoxyethylene fatty acid ester; a polyoxyethylene alkylphenyl ether; a polyoxyethylene alkyl ether; a cellulose derivative such as alkyl cellulose, hydroxyalkyl cellulose and hydroxyalkyl alkylcellulose; a carboxyvinyl polymer such as a carbomer, for example Carbopol 971 and Carbopol 974; a polyvinyl polymer; a polyvinyl alcohol; a polyvinylpyrrolidone; a copolymer of polyoxypropylene and polyoxyethylene; tyloxapol; and combinations thereof.

Examples of suitable polymers include, but are not limited to, polyethylene glycol polyethylene glycol distearate, hydroxypropyl methylcellulose, monostearate, hydroxypropylcellulose, polyvinylpyrrolidone, polyoxyethylene lauryl ether, polyoxyethylene octyldodecyl ether, polyoxyethylene stearyl ether, polyoxyethylene myristyl ether, polyoxyethylene oleyl ether, sorbitan esters, polyoxyethylene hexadecyl ether (e.g., cetomacrogol 1000), polyoxyethylene castor oil derivatives, polyoxyethylene sorbitan fatty acid esters (e.g., Tween 20 and Tween 80 (ICI Specialty Chemicals)); polyethylene glycols (e.g., Carbowax 3550 and 934 (Union Carbide)), polyoxyethylene stearates, carboxymethylcellulose calcium, carboxymethylcellulose sodium, methylcellulose, hydroxyethylcellulose, hydroxypropyl methylcellulose, cellulose, polyvinyl alcohol (PVA), poloxamers (e.g., Pluronics F68 and FI08, which are block copolymers of ethylene oxide and propylene oxide); poloxamines (e.g., Tetronic 908, also known as Poloxamine 908, which is a tetrafunctional block copolymer derived from sequential addition of propylene oxide and ethylene oxide to ethylenediamine (BASF Wyandotte Corporation, Parsippany, N.J.)); Tetronic 1508 (T-1508) (BASF Wyandotte Corporation), Tritons X-200, which is an alkyl aryl polyether sulfonate (Rohm and Haas); PEG-derivatized phospholipid, PEG-derivatized cholesterol, PEG-derivatized cholesterol derivative, PEG-derivatized vitamin A, PEG-derivatized vitamin E, random copolymers of vinyl pyrrolidone and vinyl acetate, combinations thereof and the like.

Particularly preferred examples of polymers are tyloxapol and a copolymer of polyoxypropylene and polyoxyethylene. More particularly, the copolymer of polyoxypropylene and polyoxyethylene may be a triblock copolymer comprising a hydrophilic block-hydrophobic block-hydrophilic block configuration.

In one embodiment, the eye drop formulation for use according to the present disclosure comprises a polymer which is a poloxamer. Poloxamers can include any type of poloxamer known in the art. Poloxamers include poloxamer 101, poloxamer 105, poloxamer 108, poloxamer 122, poloxamer 123, poloxamer 124, poloxamer 181, poloxamer 182, poloxamer 183, poloxamer 184, poloxamer 185, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, poloxamer 407, poloxamer 105 benzoate and poloxamer 182 dibenzoate. Poloxamers are also referred to by their trade name Pluronic such as Pluronic 10R5, Pluronic 17R2, Pluronic 17R4, Pluronic 25R2, Pluronic 25R4, Pluronic 31 R1, Pluronic F 108, Pluronic F 108, Pluronic F 108, Pluronic F 108NF, Pluronic F 127, Pluronic F 127 NF, Pluronic F 127, Pluronic F 127, Pluronic F 38, Pluronic F 38, Pluronic F 68, Pluronic F 77, Pluronic F 87, Pluronic F 88, Pluronic F 98, Pluronic L 10, Pluronic L 101, Pluronic L 121, Pluronic L 31, Pluronic L 3S, Pluronic L 43, Pluronic L 44, Pluronic L 61, Pluronic L 62, Pluronic L 62 LF, Pluronic L 620, Pluronic L 64, Pluronic L 81, Pluronic L 92, Pluronic L 44, Pluronic N 3, Pluronic P 103, Pluronic P 104, Pluronic P 85, Pluronic P 123, Pluronic P 65, Pluronic P 84, Pluronic P 85, combinations thereof and the like.

Especially useful polymers as stabilizers are poloxamers. Poloxamers can include any type of poloxamer known in the art. Poloxamers include poloxamer 101, poloxamer 105, poloxamer 108, poloxamer 122, poloxamer 123, poloxamer 124, poloxamer 181, poloxamer 182, poloxamer 183, poloxamer 184, poloxamer 185, poloxamer 188, poloxamer 212, poloxamer 215, poloxamer 217, poloxamer 231, poloxamer 234, poloxamer 23S, poloxamer 237, poloxamer 238, poloxamer 282, poloxamer 284, poloxamer 288, poloxamer 331, poloxamer 333, poloxamer 334, poloxamer 33S, poloxamer 338, poloxamer 401, poloxamer 402, poloxamer 403, poloxamer 407, poloxamer 105 benzoate and poloxamer 182 di benzoate. Poloxamers are also referred to by their trade name Pluronic such as Pluronic 10R5, Pluronic 17R2, Pluronic 17R4, Pluronic 25R2, Pluronic 25R4, Pluronic 31 R1, Pluronic F 108 Cast Solid Surfacta, Pluronic F 108 NF, Pluronic F 108 Pastille, Pluronic F 108NF Prill Poloxamer 338, Pluronic F 127, Pluronic F 127 NF, Pluronic F 127 NF 500 BHT Prill, Pluronic F 127 NF Prill Poloxamer 407, Pluronic F 38, Pluronic F 38 Pastille, Pluronic F 68, Pluronic F 68 Pastille, Pluronic F 68 LF Pastille, Pluronic F 68 NF, Pluronic F 68 NF Prill Poloxamer 188, Pluronic F 77, Pluronic F 77 Micropastille, Pluronic F 87, Pluronic F 87 NF, Pluronic F 87 NF Prill Poloxamer 237, Pluronic F 88, Pluronic F 88 Pastille, Pluronic F 98, Pluronic L 10, Pluronic L 101, Pluronic L 121, Pluronic L 31, Pluronic L 35, Pluronic L 43, Pluronic L 44 NF Poloxamer 124, Pluronic L 61, Pluronic L 62, Pluronic L 62 LF, Pluronic L 620, Pluronic L 64, Pluronic L 81, Pluronic L 92, Pluronic L44 NF INH surfactant Poloxamer 124 View, Pluronic N 3, Pluronic P 103, Pluronic P 104, Pluronic P 105, Pluronic P 123 Surfactant, Pluronic P 65, Pluronic P 84, Pluronic P 85, combinations thereof and the like. In particular, said polymer is poloxamer 407.

A further polymeric stabilizing agent compatible with the compositions and methods described herein is tyloxapol. In preferred embodiments, the stabilizer and co-solubilizer is tyloxapol, which is a 4-(1,1,3,3-tetramethylbutyl) phenol polymer with formaldehyde and oxirane.

The amount of polymer, for example poloxamer, more preferably poloxamer 407, in the eye drop formulation for use according to the present disclosure may be 0.1 to 5%, in particular 0.5 to 4%, more particularly 1 to 3%, even more particularly 2 to 3%, for example of about 2.5% by weight of polymer based on the volume of the composition.

### Additive to Prevent Oxidation of the Drug

The eye drop formulation may also comprise an additive to prevent the oxidation of the dexamethasone, for example as disclosed in WO2021/001366 and US20220354869, which content is incorporated by reference in its entirety. In a preferred embodiment, the additive to prevent the oxidation of the dexamethasone is selected from antioxidants, oxygen scavengers and mixtures thereof.

Antioxidants typically include phenolic antioxidant and reducing agent. Phenolic antioxidants are sterically hindered phenols that react with free radicals, blocking the oxidation reaction. Among phenolic antioxidants, one can cite butylated hydroxyanisole (BHA), butylated hydroxytoluene (BHT), tert-butylhydroquinone (TBHQ) or 3,4-dhydroxybenzoic acid, dodecyl 3,4,5-trihydroxybenzoate (lauryl gallate). Reducing agent are compounds that have lower redox potential than the drug they are intended to prevent from oxidation. Reducing agents scavenger oxygen from the medium and thus delay or prevent oxidation. Among reducing agents, sodium thiosulfate (STS) is an advantageous antioxidant. Examples of antioxidants further include water soluble natural antioxidants such as ascorbic acid, malic acid, citric acid, tartaric acid, lactic acid, and other organic acids and their derivatives.

In certain aspects the eye drop formulation is in a container that allows oxidation of its contents, for example a plastic container. Oculis Operations Sàrl discovered that the degradation of dexamethasone in a plastic container could be reduced or prevented by using an antioxidation agent. These antioxidant containing formulations are described in WO2021001366. Sodium thiosulfate is a particularly good antioxidant to prevent the degradation of dexamethasone in a container that allows oxidation of its contents.

In a specific embodiment, the additive to prevent the oxidation of the dexamethasone is sodium thiosulfate

In another specific embodiment, the additive to prevent the oxidation of the dexamethasone is selected among sodium thiosulfate, methionine, 3,4-dihydroxybenzoic acid, sodium citrate, malic acid, sodium ascorbate, tartaric acid, □-monothioglycerol, butylated hydroxyanisole, lauryl gallate, lactic acid, tert-butylhydroquinone, and their salts or derivatives, or mixtures thereof. More preferably, said additive is selected among sodium thiosulfate, methionine (typically L-methionine), 3,4-dihydroxybenzoic acid, sodium citrate (e.g. sodium citrate tribasic dehydrate), malic acid (typically DL-malic acid, sodium ascorbate (e.g. (+)-sodium L-ascorbate), tartaric acid (typically DL-tartaric acid), □-monothioglycerol, and butylated hydroxyanisole, and even more preferably, said additive is selected among sodium thiosulfate, methionine, and, 3,4-dihydroxybenzoic acid, Of course, a mixture of said antioxidants may be added as additive to prevent the oxidation of the drug.

The amount of additive to prevent the oxidation of the dexamethasone in the eye drop formulation may be 0 to 2%, in particular 0 to 1%, more particularly 0 to 0.8%, even more particularly 0.05 to 0.3% by weight of additive to prevent the oxidation of the dexamethasone based on the volume of the composition.

The additive to prevent the oxidation of the dexamethasone, typically sodium thiosulfate, methionine, or 3,4 dihydroxybenzoic acid, can be added at a concentration of at least 0.05% (w/v), preferably at a concentration between 0.05% (w/v) and 1% (w/v), more preferably between 0.05 to 0.5%, and still more preferably between 0.05% (w/v) and 0.4% (w/v). The additive to prevent the oxidation of the drug, typically sodium thiosulfate, can be added at a concentration between 0.05% (w/v) and 0.3% (w/v). In other aspects the additive to prevent the oxidation of the drug, typically sodium thiosulfate, is at a concentration between 0.1% and 0.6%, between 0.1% and 0.5%, between 0.2% and 0.6%, between 0.2% and 0.5%, between 0.1% and 0.4%, or between 0.2% and 0.4%. Any form of sodium thiosulfate can be used in the formulation. Examples of forms of sodium thiosulfate include amorphous, anhydrate, monohydrate, dihydrate and pentahydrate, each of which can have morphic forms. The weight used should be adjusted accordingly.

As used herein, the concentration of sodium thiosulfate refers to the weight of the water-free sodium thiosulfate. In certain embodiments the sodium thiosulfate is in the form of a pentahydrate before being dissolved in the formulation. For other stable sodium thiosulfate hydrates, for example sodium thiosulfate monohydrate and sodium thiosulfate dihydrate, as well as other antioxidants, typically the molar equivalent of 0.3% sodium thiosulfate may be used in the eye drop formulation.

In certain embodiments the concentration of sodium thiosulfate in the formulation is about 0.1%, about 0.15%, about 0.2%, about 0.25%, about 0.3%, about 0.35%, about 0.4%, about 0.45%, or about 0.5%.

In a particularly preferred embodiment, the eye drop formulation for use according to the present disclosure comprises:
−1.5% of dexamethasone;
−5 to 25% of γ-cyclodextrin, for example 12 to 16% of γ-cyclodextrin;
−2 to 2.7% of polymer, for example 2% or 2.7% of poloxamer;
−0 to 0.1% of chelating agent, for example 0.1% of chelating agent, typically, disodium edetate;
−0.05 to 0.45% of an additive to prevent the oxidation of the dexamethasone, for example between 0.05% and 0.4%, or between 0.05% and 0.3%, of an additive to prevent the oxidation of the corticosteroid, typically phenolic antioxidants or reducing agents, such as water-soluble natural antioxidants, and more preferably sodium thiosulfate, L-methionine, or 3,4-dihydroxybenzoic acid;
−0 to 1% of electrolyte, for example 0.57% of electrolyte, typically sodium chloride; and
water;
wherein the % are % by weight based on the volume of the composition.

The eye drop formulation comprising dexamethasone and γ-cyclodextrin can be stored in plastic vials, typically LDPE vials, or glass vials.

In specific embodiments, the eye drop formulation comprising dexamethasone, γ-cyclodextrin and the additive to prevent oxidation can be stored in plastic vials; such as LDPE vials.

The pH of the eye drop formulation is preferably comprised between 4.5 and 7.0, for example between 4.7 and 6.0.

Preferred eye drop formulations for use according to the present methods of treatment

In a particular embodiment, the eye drop formulation for use according to the present methods of treatment comprises or essentially consists of:
−1.5% of dexamethasone;
−5 to 25% of γ-cyclodextrin, for example 12 to 16% of γ-cyclodextrin;
−0 to 0.1% of chelating agent, for example 0.1% of disodium edetate;
−0 to 1% of electrolyte, for example 0.57% of electrolyte, typically sodium chloride; and
water;
wherein the % are % by weight based on the volume of the composition.

In a particular embodiment, an eye drop formulation for use as described in the present specification comprises or essentially consists of:
−1.5% of dexamethasone;
−5 to 25% of γ-cyclodextrin, for example 12 to 16% of γ-cyclodextrin;
−2 to 2.7% of polymer for example 2% or 2.7% of poloxamer; such as poloxamer 407;
−0 to 0.2% of chelating agent, for example 0.1% of chelating agent, typically, disodium edetate;
−0% to 0.6% of an additive to prevent the oxidation of the dexamethasone, for example between 0.0.5% and 0.45%, or between 0.05% and 0.3%, of an additive to prevent the oxidation of the corticosteroid, typically phenolic antioxidants or reducing agents, such as water-soluble natural antioxidants, and more preferably sodium thiosulfate, L-methionine, or 3,4-dihydroxybenzoic acid;
−0 to 1% of electrolyte, for example 0.57% of electrolyte, typically sodium chloride; and
water;
wherein the % are % by weight based on the volume of the composition.

In a particularly preferred embodiment, an eye drop formulation for use according to the methods of treatment described herein, comprises or essentially consists of:
−1.5% of dexamethasone;
−14% of γ-cyclodextrin;
−2.5% of poloxamer, such as poloxamer 407;
−0 to 0.2% of chelating agent, for example 0.1% of disodium edetate;
0 to 1% of electrolyte, for example 0.57% of sodium chloride;
0% to 0.6% of an additive to prevent the oxidation of the dexamethasone, for example between 0.05% and 0.3%, of an additive to prevent the oxidation of the corticosteroid, typically phenolic antioxidants or reducing agents, such as water-soluble natural antioxidants, and more preferably sodium thiosulfate, L-methionine, or 3,4-dihydroxybenzoic acid;

### And

water;
wherein the % are % by weight based on the volume of the composition.

Typically, an eye drop formulation for use according to the methods of treatment described herein, has the following components:
−1.5% of dexamethasone;
−14% of γ-cyclodextrin;
−2.5% of poloxamer, such as poloxamer 407;
0.1% of disodium edetate;
0.57% of sodium chloride; and
between 0.05% and 0.3% of sodium thiosulfate;
water;

All the above-described formulations or aqueous compositions are advantageously preservative free.

In preferred embodiments, said eye drop formulations are prepared as a microsuspension including complex aggregates of dexamethasone and γ-cyclodextrin. In specific embodiments, 60 to 95% by weight, more particularly, 70 to 90% by weight of the dexamethasone in the composition may be in the form of a solid complexes of dexamethasone and γ-cyclodextrin.

Methods for preparing such formulations comprise the steps of:
a) mixing the dexamethasone in an ophthalmically acceptable medium with the other excipients and optionally heating for sterilization;
b) suspending gamma cyclodextrin in an ophthalmically acceptable medium to form a suspension and optionally heating said suspension for sterilization;
c) mixing the compositions of step a) and b) at a temperature T1 lower than 120° C. for a time t; for example at a temperature T1 between 50° C. and 110° C., preferably between 50° C. and 95° C., for example between 8° and 95° C. and;
d) cooling the resulting solution to a temperature T2 to obtain an aqueous composition comprising a solid complex of dexamethasone and a cyclodextrin (preferably gamma cyclodextrin).

In the above manufacturing method, the dexamethasone may be suspended in an ophthalmically acceptable medium free of cyclodextrin, optionally with the other excipients. Separately γ-cyclodextrin may be suspended in an ophthalmically acceptable medium free of active pharmaceutical ingredient. The two suspensions may be heated or sterilized, for example, by heating in an autoclave for 121° C. for 20 minutes. Then the two suspensions or hot solutions may be mixed together at a temperature ranging between 80-95° C.

The resulting solution may be cooled at a rate sufficient to produce a microsuspension comprising a solid active pharmaceutical ingredient/γ-cyclodextrin complex.

Detailed methods for manufacturing the microsuspensions are also described in WO2018100434, which content is hereby incorporated in its entirety.

Such microsuspension as above-described are stable and may be used as an eye drop formulation. In specific embodiments, said eye drop formulation with 1.5% dexamethasone (w/v) are ophthalmic microsuspensions, preservative-free. They may be presented in unit doses of 0.3 mL fill volume, for example in LDPE plastic material. The resulting suspension may be stored at ambient temperature, below 25° C., and stored for at least 2, 3, 6, 12, 18 or 24 months or even 3 years.

### Use of the Eye Drop Formulation Comprising Dexamethasone

The eye drop formulations are prepared for their use in the treatment of diabetic macular edema (DME) according to the methods with the particular dosing regimen disclosed herein. More specifically, the eye drop formulation comprising dexamethasone as described herein is topically administered to an affected eye in a human subject in need thereof according to a dosing regimen which comprises:
(i) an induction phase of topically administering to an affected eye of said subject, an eye drop formulation comprising 1.5% (w/v) dexamethasone, at a dosing of 4 to 6 drops a day, followed by,
(ii) a maintenance phase at a daily dosing less frequent than the induction phase.
   The objective of the induction phase is to provide a rapid clinical benefit for patients suffering from diabetic macular edema in terms of reduction of the symptoms associated with diabetic macular edema, in particular an improvement of the visual acuity.

The induction phase is preferably with a dosing of five, six, or seven drops a day, for a period ranging from 5 days, 1 week, 2 weeks, 3 weeks, 4 weeks, 5 weeks and up to 6 weeks, preferably 6 weeks.

The objective of the maintenance phase is to provide maintenance of the benefits acquired at the end of the induction phase while minimizing the amount of dexamethasone daily administered to the patient, being at least a daily dosing less frequent than the induction phase. The maintenance phase may be as long as required for the patient, and is preferably with a daily dosing of one, two, or three drops a day, for example three drops a day, for at least 4, 5, 6, 7, 8, 9, 10, 11, 12, 18, 24, 36, 40, 46 weeks, or indefinitely.

In a specific embodiment, the induction phase comprises topically administering to an affected eye of said subject, an eye drop formulation comprising 1.5% (w/v) dexamethasone at a dosing of 5-7 drops a day for a period ranging from 5 days and up to 6 weeks.

In another embodiment, the induction phase comprises topically administering to an affected eye of said subject, an eye drop formulation comprising 1.5% (w/v) dexamethasone at a dosing of 6 drops a day.

In certain aspects, the maintenance phase comprises topically administering to an affected eye of said subject, an eye drop formulation comprising 1.5% (w/v) dexamethasone at a dosing of 1 to 3 drops a day.

In certain aspects, the maintenance phase comprises topically administering to an affected eye of said subject, an eye drop formulation comprising 1.5% (w/v) dexamethasone at a dosing of 3 drops a day.

In a preferred embodiment, the dosing regimen comprises:
(i) an loading phase of topically administering to an affected eye of said subject, an eye drop formulation comprising 1.5% (w/v) dexamethasone, at a dosing of 6 drops a day, for 6 weeks, followed by
(ii) a maintenance phase of topically administering said eye drop formulation at a dosing of 3 drops a day.

In a particularly preferred embodiment, the dosing regimen comprises:
(i) an induction phase of topically administering to an affected eye of said subject, an eye drop formulation comprising 1.5% (w/v) dexamethasone, at a dosing of 6 drops a day for 6 weeks, followed by
(ii) a maintenance phase of topically administering said eye drop formulation at a dosing of 3 drops a day for at least 6 weeks, for example, at least 46 weeks, and as long as needed for the patient.

The eye drop formulations of the present disclosure have been tested in clinical trials in patients suffering from such diabetic macular edema and the results are provided in the Examples. In particular, efficacy has been shown for treating diabetic macular edema with eye drop formulation of 1.5% (w/v) dexamethasone thanks to its specific dosing regimen with an induction phase and maintenance phase as described above.

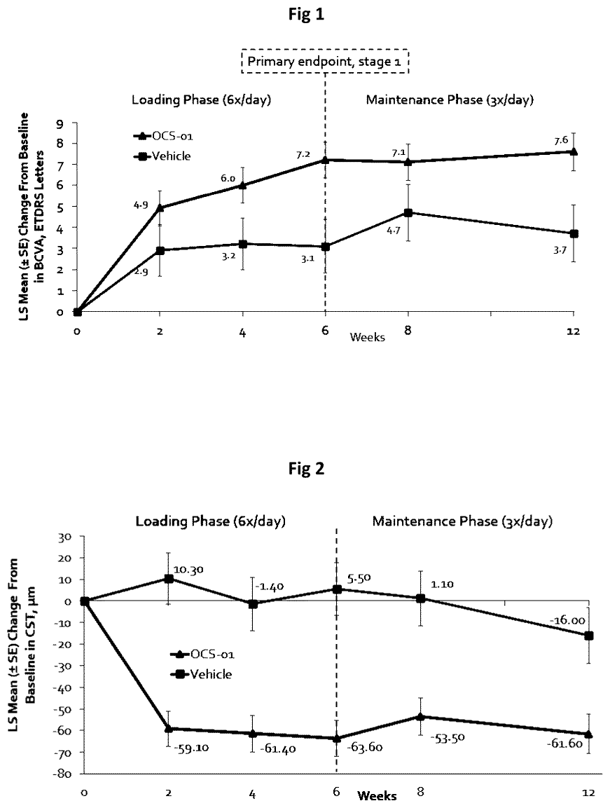

Human clinical trials described further in Example 2 have shown that the use of an induction dose of 6 drops a day provided a strong and fast improvement in efficacy as shown by
a mean change at week 6 of 7.2 BCVA/ERDTS letter score from baseline,
a mean change in central subfield thickness (CST) as assessed by optical coherence tomography, at the end of the induction phase at week 6 was −63.6 mm from baseline
25.3% of the patients reaching more than 3 line (15 letter) gain in BCVA,
The maintenance dose of 3 drops a day was sufficient to maintain such improvements (and even increase) in visual acuity at week 12 reaching a mean change of 7.6 BCVA/ERDTS letters compared to baseline (see FIG. 1) and a mean change of CST of −61.6 mm at week 12 from baseline.
In addition, no unexpected safety findings were observed at week 12.

In specific embodiments, the mean ETDRS Best Corrected Visual Acuity is improved at the end of the induction phase of such above treatment in the patients suffering from DME, for example to at least 4, 5, or 6 ETDRS letters as measured from baseline, preferably at least 7 ETDRS letters as measured from baseline, ETDRS Best Corrected Visual Acuity being determined as described in the Examples below.

In an embodiment, the mean ETDRS Best Corrected Visual Acuity is maintained during the maintenance phase to at least 6 ETDRS letters as measured from baseline, preferably at least 7 ETDRS letters as measured from baseline.

In a preferred embodiment, the mean ETDRS Best Corrected Visual Acuity is maintained to at least 6 ETDRS letters as measured from baseline, preferably at least 7 ETDRS letters as measured from baseline, after 6 weeks of maintenance phase. In specific embodiments, the LS mean change of the central subfield thickness (CST) is reduced to more than −50 μm CST as measured from baseline, preferably more than −60 μm CST as measured from baseline, after 6 weeks of induction phase in said subjects suffering from DME. In an embodiment, the reduction of the LS mean change of the central subfield thickness (CST) is maintained to more than −50 μm CST as measured from baseline during the maintenance phase, preferably after 6 weeks of maintenance phase.

In some embodiments, the administration regime described herein results in an improved BCVA/ETDRS letter assessment across a population of treated patients of at least 3 lines gained (≥15 letters) at 12 months from baseline of at least 25%, at least 27%, at least 30%, at least 35%, or greater than 35%.

In a preferred embodiment of the method, the eye drop formulation for use in the above method, comprises or essentially consists of:
−1.5% of dexamethasone;
−14% of γ-cyclodextrin;
−2.5% of poloxamer, such as poloxamer 407;
−0 to 0.2% of chelating agent, for example 0.1% of disodium edetate;
−0 to 1% of electrolyte, for example 0.57% of sodium chloride;
−0% to 0.6% of an additive to prevent the oxidation of the dexamethasone, for example between 0.05% and 0.45%, or between 0.05% and 0.3%, of an additive to prevent the oxidation of the corticosteroid, typically sodium thiosulfate;

### And

water;
wherein the % are % by weight based on the volume of the composition.

In specific embodiments, the present treatment is useful in subjects suffering from DME in need of intravitreal injectable drug treatment in order to reduce or eliminate problems associated with such intravitreal injectable drug treatment. Such treatments include without limitations, anti-VEGF drugs, such as for example ranibizumab (Lucentis; Genentech, South San Francisco, CA, USA), approved in August 2012, and aflibercept (Eylea; Regeneron Pharmaceuticals, Tarrytown, NY, USA), approved in July 2014.

The present treatment is particularly useful for patients with no or inadequate response to VEGF inhibitor treatments (VEGFi naïve patients) and/or which do not support invasive treatments for diabetic macular edema.

Hence, in a particular embodiment of the above method for treating diabetic macular edema, the patient is selected among VEGFi naïve patients, with retinal thickening in the affected eye due to diabetic macular edema.

Typically, the patient is a human patient, and more specifically an adult human patient.

### SPECIFIC EMBODIMENTS

Embodiment E1: A method of treating diabetic macular edema in a human subject in need thereof, said method comprising
(i) an induction phase of topically administering to an affected eye of said subject, an eye drop formulation comprising 1.5% (w/v) dexamethasone, at a dosing of 5 to 7 drops a day, followed by,
(ii) a maintenance phase of topically administering to an affected eye of said subject, said eye drop formulation comprising 1.5% (w/v) dexamethasone at a daily dosing less frequent than the induction phase.

Embodiment E2: The method of Embodiment E1, wherein the eye drop formulation is a microsuspension comprising solid complexes of dexamethasone, gamma-cyclodextrin and poloxamer.

Embodiment E3: The method of Embodiment E1 or E2, wherein the eye drop formulation is a microsuspension comprising microparticles of a diameter D50 ranging from about 1 mm to 90 mm.

Embodiment E4: The method of any one of Embodiments E1-E3, wherein the induction phase comprises topically administering to an affected eye of said subject, an eye drop formulation comprising 1.5% (w/v) dexamethasone at a dosing of 5 to 7 drops a day for a period ranging from 5 days and up to 6 weeks.

Embodiment E5: The method of any one of Embodiments E1-E4, wherein the induction phase comprises topically administering to an affected eye of said subject, an eye drop formulation comprising 1.5% (w/v) dexamethasone at a dosing of 5 to 7 drops a day for a period of 6 weeks.

Embodiment E6: The method of any one of Embodiments E1-E4, wherein the induction phase comprises topically administering to an affected eye of said subject, an eye drop formulation comprising 1.5% (w/v) dexamethasone at a dosing of 6 drops a day for a period of 6 weeks.

Embodiment E7: The method of any one of Embodiments E1-E5, wherein the maintenance phase comprises topically administering to an affected eye of said subject, an eye drop formulation comprising 1.5% (w/v) dexamethasone at a dosing of 1, 2 or 3 drops a day.

Embodiment E8: The method of any one of Embodiments E1-E7, wherein the maintenance phase comprises topically administering to an affected eye of said subject, an eye drop formulation comprising 1.5% (w/v) dexamethasone at a dosing of 3 drops a day.

Embodiment E9: The method of any one of Embodiments E1-E8, wherein the dosing regimen comprises
(i) an induction phase of topically administering to an affected eye of said subject, an eye drop formulation comprising 1.5% (w/v) dexamethasone, at a dosing of 6 drops a day, followed by
(ii) a maintenance phase of topically administering said eye drop formulation at a dosing of 1, 2 or 3 drops a day for at least 6 weeks.

Embodiment E10: The method of any one of Embodiments E1-E9, wherein the dosing regimen comprises
(i) an induction phase of topically administering to an affected eye of said subject, an eye drop formulation comprising 1.5% (w/v) dexamethasone, at a dosing of 6 drops a day for 6 weeks, followed by
(ii) a maintenance phase of topically administering said eye drop formulation at a dosing of 3 drops a day.

Embodiment E11: The method of any one of Embodiments E1-E10, wherein the dosing regimen comprises
(i) an induction phase of topically administering to an affected eye of said subject, an eye drop formulation comprising 1.5% (w/v) dexamethasone, at a dosing of 6 drops a day for 6 weeks, followed by
(ii) a maintenance phase of topically administering said eye drop formulation at a dosing of 3 drops a day, wherein said eye drop formulation is a microsuspension comprising solid complexes of dexamethasone, gamma-cyclodextrin and poloxamer.

Embodiment E12: The method of any one of Embodiments E1-E11, wherein the eye drop formulation comprises an amount of gamma-cyclodextrin from 12 to 16%, by weight of cyclodextrin based on the volume of the composition.

Embodiment E13: The method of any one of Embodiments E1-E12, wherein the dosing regimen comprises
(i) an induction phase of topically administering to an affected eye of said subject, an eye drop formulation comprising 1.5% (w/v) dexamethasone, at a dosing of 6 drops a day for 6 weeks, followed by
(ii) a maintenance phase of topically administering said eye drop formulation at a dosing of 3 drops a day,
wherein said eye drop formulation is a microsuspension comprising solid complexes of dexamethasone and gamma-cyclodextrin, said microsuspension comprises or essentially consists of:
−1.5% of dexamethasone;
−12% to 16% of γ-cyclodextrin;
−2% to 2.7% of a polymer;
−0 to 0.1% of chelating agent, for example 0.1% of disodium edetate;
−0 to 1% of electrolyte, for example 0.57% of sodium chloride;
−0% to 0.6% of an additive to prevent the oxidation of the dexamethasone, and
water;
the pH is between 4.7 and 6.0,
wherein the % are % by weight based on the volume of the composition.

Embodiment E14: The method of any one of embodiments E1 to E13, wherein the maintenance phase comprises topically administering said eye drop formulation at a dosing of 3 drops a day for at least 6 or at least 12 weeks.

Embodiment E15: The method of any one of embodiments E1 to E14, wherein the maintenance phase comprises topically administering said eye drop formulation at a dosing of 3 drops a day for at least 46 weeks.

Embodiment E16: The method of any one of embodiments E1 to E15, wherein the maintenance phase comprises topically administering said eye drop formulation at a dosing of 3 drops a day for a period of time as long as needed for the patient.

Embodiment E17: The method of any one of embodiments E1 to E16, wherein the maintenance phase comprises topically administering said eye drop formulation at a dosing of 3 drops a day indefinitely.

Embodiment E18: The method of any one of embodiments E1 to E17, wherein the maintenance phase comprises topically administering said eye drop formulation at a dosing of 3 drops a day for a certain period of time followed by a reduction of the daily dosing.

Embodiment E19: The method of any one of Embodiments 1-18, wherein said eye drop formulation, comprises or essentially consists of:
−1.5% of dexamethasone;
−14% of γ-cyclodextrin;
−2.5% of poloxamer;
−0 to 0.1% of chelating agent, for example 0.1% of disodium edetate;
−0 to 1% of electrolyte, for example 0.57% of sodium chloride;
−0% to 0.6% of an additive to prevent the oxidation of the dexamethasone, for example 0.05% to 0.3% of sodium thiosulfate, and
water;
wherein the % are % by weight based on the volume of the composition.

Embodiment E20: The method of any one of Embodiments E1-E19, wherein said subject have no or inadequate response to VEGF inhibitor treatments and/or do not support invasive treatments for diabetic macular edema.

Embodiment E21: The method of any one of Embodiments E1-E20, wherein said subject is selected among VEGFi naïve patients, with retinal thickening in the affected eye due to diabetic macular edema.

Embodiment E22: The method of any one of Embodiments E1-E21, wherein said subject is an adult human subject.

Embodiment E23: The method of any one of Embodiments E1-E22, wherein the mean ETDRS Best Corrected Visual Acuity in subjects suffering from DME is improved to at least 6 ETDRS letters at the end of the induction phase as compared to baseline.

Embodiment E24: The method of any one of Embodiments E1-E23, wherein the mean ETDRS Best Corrected Visual Acuity in subjects suffering from DME is maintained to at least 6 ETDRS letters after 6 weeks of maintenance phase as compared to baseline.

Embodiment E25: The method of any one of Embodiments E1-E24, wherein LS mean change of the central subfield thickness (CST) in subjects suffering from DME is reduced to more than −50 mm CST as measured from baseline, after 6 weeks of treatment in said subject suffering from DME.

Embodiment E26: The method of any one of Embodiments E1-E25, wherein an improved ETDRS letter assessment of at least 3 lines (15 letters) gained at 12 months from baseline occurs in at least 25%, at least 27%, at least 30%, at least 35% or greater than 35% of a population of treated patients.

Embodiment E27: The method of any one of Embodiments E1-E26, wherein the percentage of subjects with increased intraocular pressure exceeding 10 mm Hg after 12 weeks of treatment does not exceed 20%.

Embodiment E28: The method of any one of Embodiments E1-E27, wherein the percentage of subjects with increased intraocular pressure exceeding 35 mm Hg after 12 weeks of treatment does not exceed 5%.

Embodiment E29: The method of any one of Embodiments E1-E28, wherein said eye drop formulation is preservative free.

Embodiment E30: An eye drop formulation for use in a method of treating diabetic macular edema in a human subject in need thereof, said method comprising
(i) an induction phase of topically administering to an affected eye of said subject, an eye drop formulation comprising 1.5% (w/v) dexamethasone, at a dosing of 5 to 7 drops a day, followed by,
(ii) a maintenance phase of topically administering to an affected eye of said subject, said eye drop formulation comprising 1.5% (w/v) dexamethasone at a daily dosing less frequent than the induction phase.

Embodiment E31: The eye drop formulation for use of Embodiment E30, wherein the eye drop formulation is a microsuspension comprising solid complexes of dexamethasone, gamma-cyclodextrin and poloxamer.

Embodiment E32: The eye drop formulation for use of Embodiment E30 or E31, wherein the eye drop formulation is a microsuspension comprising microparticles of a diameter D50 ranging from about 1 mm to 90 mm.

Embodiment E33: The eye drop formulation for use of any one of Embodiments E30-E32, wherein the induction phase comprises topically administering to an affected eye of said subject, an eye drop formulation comprising 1.5% (w/v) dexamethasone at a dosing of 5-7 drops a day for a period ranging from 5 days and up to 6 weeks.

Embodiment E34: The eye drop formulation for use of any one of Embodiments E30-E33, wherein the induction phase comprises topically administering to an affected eye of said subject, an eye drop formulation comprising 1.5% (w/v) dexamethasone at a dosing of 5-7 drops a day for a period of 6 weeks.

Embodiment E35: The eye drop formulation for use of any one of Embodiments E30-E34, wherein the induction phase comprises topically administering to an affected eye of said subject, an eye drop formulation comprising 1.5% (w/v) dexamethasone at a dosing of 6 drops a day for a period of 6 weeks.

Embodiment E36: The eye drop formulation for use of any one of Embodiments E30-E35, wherein the maintenance phase comprises topically administering to an affected eye of said subject, an eye drop formulation comprising 1.5% (w/v) dexamethasone at a dosing of 1, 2 or 3 drops a day.

Embodiment E37: The eye drop formulation for use of any one of Embodiments E30-E36, wherein the maintenance phase comprises topically administering to an affected eye of said subject, an eye drop formulation comprising 1.5% (w/v) dexamethasone at a dosing of 3 drops a day.

Embodiment E38: The eye drop formulation for use of any one of Embodiments E29-E36, wherein the dosing regimen comprises
(i) an induction phase of topically administering to an affected eye of said subject, an eye drop formulation comprising 1.5% (w/v) dexamethasone, at a dosing of 6 drops a day, followed by
(ii) a maintenance phase of topically administering said eye drop formulation at a dosing of 1, 2 or 3 drops a day for at least 6 weeks.

Embodiment E39: The eye drop formulation for use of any one of Embodiments E30-E38, wherein the dosing regimen comprises
(i) an induction phase of topically administering to an affected eye of said subject, an eye drop formulation comprising 1.5% (w/v) dexamethasone, at a dosing of 6 drops a day for 6 weeks, followed by
(ii) a maintenance phase of topically administering said eye drop formulation at a dosing of 3 drops a day.

Embodiment E40: The eye drop formulation for use of any one of Embodiments E30-E39, wherein the dosing regimen comprises
(i) an induction phase of topically administering to an affected eye of said subject, an eye drop formulation comprising 1.5% (w/v) dexamethasone, at a dosing of 6 drops a day for 6 weeks, followed by
(ii) a maintenance phase of topically administering said eye drop formulation at a dosing of 3 drops a day,
wherein said eye drop formulation is a microsuspension comprising solid complexes of dexamethasone, gamma-cyclodextrin and poloxamer.

Embodiment E41: The eye drop formulation for use of any one of Embodiments E30-E40, wherein the eye drop formulation comprises an amount of gamma-cyclodextrin from 12 to 16%, by weight of cyclodextrin based on the volume of the composition.

Embodiment E42: The eye drop formulation for use of any one of Embodiments E30-E41, wherein the dosing regimen comprises
(i) an induction phase of topically administering to an affected eye of said subject, an eye drop formulation comprising 1.5% (w/v) dexamethasone, at a dosing of 6 drops a day for 6 weeks, followed by
(ii) a maintenance phase of topically administering said eye drop formulation at a dosing of 3 drops a day,
wherein said eye drop formulation is a microsuspension comprising solid complexes of dexamethasone and gamma-cyclodextrin, said microsuspension comprises or essentially consists of:
−1.5% of dexamethasone;
−12% to 16% of γ-cyclodextrin;
−2% to 2.7% of a polymer;
−0 to 0.1% of chelating agent, for example 0.1% of disodium edetate;
−0 to 1% of electrolyte, for example 0.57% of sodium chloride;
−0% to 0.6% of an additive to prevent the oxidation of the dexamethasone, and
water;
the pH is between 4.7 and 6.0,
wherein the % are % by weight based on the volume of the composition.

Embodiment E43: The eye drop formulation for use of any one of embodiments E30 to E42, wherein the maintenance phase comprises topically administering said eye drop formulation at a dosing of 3 drops a day for at least 6 or at least 12 weeks.

Embodiment E44: The eye drop formulation for use of any one of embodiments E30 to E43, wherein the maintenance phase comprises topically administering said eye drop formulation at a dosing of 3 drops a day for at least 46 weeks.

Embodiment E45: The eye drop formulation for use of any one of embodiments E30 to E44, wherein the maintenance phase comprises topically administering said eye drop formulation at a dosing of 3 drops a day for a period of time as long as needed for the patient.

Embodiment E46: The eye drop formulation for use of any one of embodiments E30 to E45, wherein the maintenance phase comprises topically administering said eye drop formulation at a dosing of 3 drops a day indefinitely.

Embodiment E47: The eye drop formulation for use of any one of embodiments E30 to E46, wherein the maintenance phase comprises topically administering said eye drop formulation at a dosing of 3 drops a day for a certain period of time followed by a reduction of the daily dosing.

Embodiment E48: The eye drop formulation for use of any one of Embodiments E30-E47, wherein said eye drop formulation, comprises or essentially consists of:
−1.5% of dexamethasone;
−14% of γ-cyclodextrin;
−2.5% of poloxamer;
−0 to 0.1% of chelating agent, for example 0.1% of disodium edetate;
0 to 1% of electrolyte, for example 0.57% of sodium chloride;
0% to 0.6% of an additive to prevent the oxidation of the dexamethasone, for example 0.05% to 0.3% of sodium thiosulfate, and
water;
wherein the % are % by weight based on the volume of the composition.

Embodiment E49: The eye drop formulation for use of any one of Embodiments E30-E48, wherein said subject have no or inadequate response to VEGF inhibitor treatments and/or do not support invasive treatments for diabetic macular edema.

Embodiment E50: The eye drop formulation for use of any one of Embodiments E30-E49, wherein said subject is selected among VEGFi naïve patients, with retinal thickening in the affected eye due to diabetic macular edema.

Embodiment E51: The eye drop formulation for use of any one of Embodiments E30-E50, wherein said subject is an adult human subject.

Embodiment E52: The eye drop formulation for use of any one of Embodiments E30-E51, wherein the mean ETDRS Best Corrected Visual Acuity in subjects suffering from DME is improved to at least 6 ETDRS letters at the end of the induction phase as compared to baseline.

Embodiment E53: The eye drop formulation for use of any one of Embodiments E30-E52, wherein the mean ETDRS Best Corrected Visual Acuity in subjects suffering from DME is maintained to at least 6 ETDRS letters after 6 weeks of maintenance phase as compared to baseline.

Embodiment E54: The eye drop formulation for use of any one of Embodiments E30-E53, wherein LS mean change of the central subfield thickness (CST) in subjects suffering from DME is reduced to more than −50 mm CST as measured from baseline, after 6 weeks of treatment in said subject suffering from DME.

Embodiment E55: The method of any one of Embodiments E30-E54, wherein an improved ETDRS letter assessment of at least 3 lines (15 letters) gained at 12 months from baseline occurs in at least 25%, at least 27%, at least 30%, at least 35% or greater than 35% of a population of treated patients.

Embodiment E56: The eye drop formulation for use of any one of Embodiments E30-E55, wherein the percentage of subjects with increased intraocular pressure exceeding 10 mm Hg after 12 weeks of treatment does not exceed 20%.

Embodiment E57: The eye drop formulation for use of any one of Embodiments E30-E56, wherein the percentage of subjects with increased intraocular pressure exceeding 35 mm Hg after 12 weeks of treatment does not exceed 5%.

Embodiment E58: The eye drop formulation for use of any one of Embodiments E30-E57, wherein said eye drop formulation is preservative free.

### Examples

### OCS-01 Eye Drops Formulation

Formulation of dexamethasone eye drops having a composition according to table below were prepared.

| Ingredients | Quantity (% w/v) |
|---|---|
| Dexamethasone | 1.500 |
| γ-cyclodextrin | 14.000 |
| Disodium edetate dihydrate | 0.100 |
| Poloxamer 407 | 2.500 |
| Sodium chloride | 0.570 |
| Sodium Thio sulfate | 0.471 |
| pentahydrate | |
| Water for injection | Q.S. for 100.00 |

The formulation for the examples is prepared as described below:

The manufacturing process is divided into two parts, A and B, that compose the final drug product bulk. Part A contains Water for injection, Sodium chloride, Disodium Edetate Dihydrate, Poloxamer 407, Sodium Thiosulfate Pentahydrate and Dexamethasone, while part B contains Water for injection and Gamma Cyclodextrin. Each suspension is sterilized by heat, whereafter part B is aseptically transferred into the tank containing Part A. This resulting solution is then cooled down to ambient temperature under gentle stirring. Then, the bulk product is aseptically filled into single-dose units by Blow-Fill-Seal technology.

### Example 1: Phase I Clinical Trial—a Prospective, Single-Center, Open-Label, Study of the Plasma Pharmacokinetics and Safety Following Topical Administration of OCS-01 Ophthalmic Suspension in Healthy Adult Subjects-Study DX217

### Background and Rationale

A prospective, single-center, open-label, Phase 1 study was designed to evaluate the plasma pharmacokinetics (PK) and safety profile of OCS-01 Ophthalmic Suspension following topical ocular administration for two days in the study eye using either 1 Time per Day, 3 Times per Day, or 6 Times per Day dosing regimens in healthy adult subjects. This clinical trial utilized entry criteria intended to appropriately identify healthy subjects while minimizing concomitant therapies, procedures, or medical conditions that could possibly put a subject at risk or otherwise interfere with study parameters. Subjects were queried as to potential adverse events (AEs), and ocular safety measures were assessed at every study visit.

In the present clinical study, OCS-01 Ophthalmic Suspension was composed of dexamethasone (1.5% weight per volume [w/v]), γ-cyclodextrin, sodium thiosulfate pentahydrate, disodium edetate, poloxamer 407, sodium chloride, and water for injection. Dexamethasone is a corticosteroid that has been used in the form of eye drops (Maxidex®, Alcon) to treat inflammation caused by surgery, infections, or injury. OCS-01 Ophthalmic Suspension is a novel drug product formulation consisting of nanoparticle aggregates of the active ingredient dexamethasone and γ-cyclodextrin, which markedly increases the solubility of the drug substance and delivers high concentrations of dexamethasone to the anterior and posterior segment target tissues following topical ocular administration.

### Objective

To characterize the plasma pharmacokinetics and safety profile of OCS-01 Ophthalmic Suspension following topical ocular administration for two days in the study eye using either 1 Time per Day, 3 Times per Day, or 6 Times per Day dosing regimens in healthy adult subjects.

### Methodology

This study was a prospective, single-center, open-label, Phase 1 study evaluating the plasma pharmacokinetics and safety profile of OCS-01 Ophthalmic Suspension following topical ocular administration for two days in the study eye using either 1 Time per Day, 3 Times per Day, or 6 Times per Day dosing regimens in healthy adult subjects.

### Number of Subjects (Planned and Analyzed)

A total of 18 healthy adult subjects were planned to be assigned to one of three treatment groups following a 1:1:1 assignment ratio: 1 Time per Day, 3 Times per Day, or 6 Times per Day dosing regimens of OCS-01 Ophthalmic Suspension. A total of 53 subjects were screened and 19 healthy adult subjects were enrolled in the study and assigned to one of the three treatment groups: 6 subjects were assigned to receive 1 Time per Day dosing, 6 subjects were assigned to receive 3 Times per Day dosing, and 7 subjects were assigned to receive 6 Times per Day dosing regimens of OCS-01 Ophthalmic Suspension.

### Test Product/Control Product, Dose, Mode of Administration

OCS-01 ophthalmic suspension (1.5% dexamethasone) was administered topically in the study eye by trained study staff in the office for 2 days. Subjects were assigned to one of three treatment arms and received a single drop (approximately 30 μL, equivalent to 0.45 mg dexamethasone per drop) of OCS-01 either 1 Time per Day, 3 Times per Day, or 6 Times per Day. This dosing regimen was equivalent to 0.45 mg, 1.35 mg, or 2.7 mg of dexamethasone per day and 0.9 mg, 2.7 mg, or 5.4 mg of dexamethasone total over the study course, respectively. No control products were used in this study.

### Duration of Treatment

An individual subject's participation in the study ranged between 6 to 31 days with a treatment duration of 2 days.

### Endpoints

### Pharmacokinetic Measures

OCS-01 is a 1.5% w/v suspension which is equivalent to 1.5 g dexamethasone per 100 mL suspension. A singledrop from an eye drop bottle is approximately 30 μL, containing approximately 0.45 mg of dexamethasone. The PK of OCS-01 was evaluated based on plasma dexamethasone concentrations.

Serial plasma samples after the first dose on Day 1 and the last dose on Day 2 were obtained to determine the pharmacokinetics of dexamethasone after administration of OCS-01 ophthalmic suspension. For each subject in the three treatment groups, the following plasma pharmacokinetic parameters were calculated whenever possible: Cmax (Maximal plasma concentration), Tmax (Corresponding time to reach maximal plasma concentration), Tlast (Timepoint with the last measurable plasma concentration), Ke (Apparent elimination rate constant estimated by a log-linear regression of the terminal phase of the concentration versus time profile), T1/2 (Apparent elimination half-life; calculated as In (2)/Ke), AUC0-∞ (Area under the plasma concentration time curve from 0 to time infinity; calculated using the linear up/log down trapezoidal rule), AUCO-3 (Area under the plasma concentration time curve from 0 to 3 hours; calculated using the linear up/log down trapezoidal rule), AUCO-6 (Area under the plasma concentration time curve from 0 to 6 hours; calculated using the linear up/log down trapezoidal rule), AUCO-24 (Area under the plasma concentration time curve from 0 to 24 hours; calculated using the linear up/log down trapezoidal rule), AUClast (AUC to the last measurable concentration), ARCmax (Accumulation ratio of Cmax; calculated as Day 2 last dose Cmax/Day 1 first dose Cmax), AR_{AUC} (Accumulation ratio of AUC; calculated as 1 Time per Day ARAUC: Day 2 AUCO-24/Day 1 AUC0-24; 3 Times per Day ARAUC: Day 2 last dose AUCO-6/Day 1 first dose AUCO-6; 6 Times per Day ARAUC: Day 2 last dose AUCO-3/Day 1 first dose AUCO-3), CL/F (Clearance divided by the bioavailable fraction), and Vz/F (Volume of distribution based on the terminal phase divided by the bioavailable fraction).

### Safety Measures

Adverse events (reported, elicited, and observed);
Best Corrected Visual Acuity (BCVA) at distance;
Slit lamp biomicroscopy;
Physical examination;
Vital signs (resting blood pressure and heart rate);
Dilated ophthalmoscopy;
Intraocular pressure (IOP); and
Hematology, blood chemistry analysis, and urinalysis.

### Other Measures

Urine pregnancy test;
Urine test for illicit drugs (including alcohol, amphetamines, barbiturates, benzodiazepines, cocaine (metabolite), cannabinoids, methadone, methamphetamine, opiates, and phencyclidine); and
Blood test for alcohol, hepatitis B surface antigen, hepatitis C antibody, antigens and antibodies to HIV types 1 and 2.

### Results

There were no substantial differences in the mean concentration-time profiles between the three treatment groups following the first dose. In contrast, the mean dexamethasone concentrations after the last dose increased with increasing dosing frequency (ie, 6 Times per Day concentrations were greater than 3 Times per Day concentrations were greater than 1 Time per Day concentrations). Inter-subject variability in plasma dexamethasone concentrations after ocular administration of OCS-01 was high (CV % ranging from 31.3 to 119.2%). This is consistent with generally higher variability observed in PK studies when dexamethasone is administered topically compared to systemically, as well as the small sample size within each treatment group.

There was no obvious effect of repeat dosing or dosing regimen on the Tmax or T1/2 of dexamethasone. The mean (SD) T1/2 after the last dose was 5.97 (1.91), 5.74 (1.33), and 6.23 (1.28) hours in the 1 Time per Day, 3 Times per Day, and 6 Times per Day groups, respectively. Although the mean Cmax and AUCO-3 values after the first dose were not consistent between all three cohorts, the range of these parameter values were overlapping between the cohorts. Any differences in the mean plasma exposure observed between cohorts after the first dose were likely due to the high inter-subject variability observed in all three cohorts.

In subjects that received OCS-01 1 Time per Day, there was little to no accumulation of dexamethasone after two doses (mean (SD) ARCmax and ARAUC were 1.00 (0.48) and 1.04 (0.40), respectively). However, accumulation based on mean Cmax and AUCO-6, or AUCO-3 values was observed with 3 Times per Day and 6 Times per Day dosing. The mean (SD) ARCmax and ARAUC values were 1.70 (0.60) and 1.94 (0.78) in the 3 Times per Day cohort and 4.18 (3.04) and 6.28 (4.57) in the 6 Times per Day cohort.

This suggests that there is increased accumulation of dexamethasone as the dose frequency of OSC-01 increases. Based on the T1/2 of dexamethasone, steady-state concentrations are expected to be achieved by Day 3 after repeat dosing. The average subject (71.4 kg) was expected to have a total volume of approximately 3060 mL of plasma, and the maximum possible plasma concentration of dexamethasone after the administration of a single drop (0.45 mg dexamethasone) was 147 ng/mL. Using the average Cmax after the first OSC-01 dose on Day 1 across all dosing groups of 794 μg/mL, approximately 0.54% of the theoretical maximum concentration (assuming 100% bioavailability) was present in the plasma at Tmax.

### Discussion and Overall Conclusions

### OCS-01 Pharmacokinetics

There was no effect of dosing regimen on the Tmax or T1/2 of dexamethasone. Little accumulation of dexamethasone was observed in the subjects that received OCS-01 1 Time per Day, however, accumulation with repeat dosing was observed in the 3 Times and 6 Times per Day cohorts. This suggests that there is incremental accumulation of dexamethasone as the dose frequency of OSC-01 increases.

Bioavailability of Dexamethasone after Ocular Administration was Approximately 0.54% of the Theoretical Maximum Concentration Present in the Plasma at Tₘₐₓ.

For comparison, the systemic bioavailability of topically applied 1% atropine eye drops was determined to be 63.5±28.6% (Timo Kaila, Juha-Matti Korte and K. Matti Saari, Acta Ophthalmol. Scand. 1999:77: 193-196). In general, only a small fraction (<5%) of topically applied drugs is absorbed into the eye while up to 80% enter the general blood circulation (Marc Labetoulle, Éric Frau, Claire Le Jeune, Systemic adverse effects of topical ocular treatments, Presse Med 2005; 34:589-95).

Hence, the present study proves that surprisingly low amount of dexamethasone entered the blood circulation thereby reducing the risk of safety issues, even at 6 drops a day despite the high amount of dexamethasone administered to the eye.

Comparisons with Published Pharmacokinetic Results for Dexamethasone

Dexamethasone plasma Cₘₐₓ values after the first and last OSC-01 doses were compared to those reported by other investigators following dexamethasone dosing by various routes. These data are presented in the following clinical study report.

Maximum dexamethasone plasma concentrations after single or multiple dosing of OCS-01 were low compared to those after oral, intramuscular (IM), or intravenous (IV) administration: Cₘₐₓ values after single and multiple-dose OCS-01 administration were, respectively, 1% to 11% and 1% to 25% those following single 0.5 to 6 mg oral doses; 2% to 3% and 2% to 6% those following a single 3 mg intramuscular dose; and 0.1% to 0.2% and 0.1% to 0.4% those after a single 6.7 mg bolus intravenous dose.

Compared to a single, bilateral otic administration of Ciprodex® (ciprofloxacin 0.3%/dexamethasone 0.1%) to pediatric patients, maximum dexamethasone plasma concentrations in this study were 21% to 44% lower after a single dose or once daily administration of OCS-01, and were 21% to 79% greater after multiple, 3-times and 6-times daily OCS-01 dosing.

Comparing single dose administration of ocular formulations, dexamethasone Cₘₐₓ values after an OCS-01 dose (dexamethasone 1.5%) were 1.6 to 2.2 times higher than after a single bilateral dose of Tobradex® (tobramycin 0.3%/dexamethasone 0.1%) and, for multiple dose administration, dexamethasone Cₘₐₓ values after two days of once daily, 3-times daily, and 6-times daily OCS-01 administration were 1.5 to 3.6 times higher than after two days of 4-times daily bilateral ocular dosing of Tobradex®. When adjusted for the dexamethasone, dose OCS-01 results in about 60% lower dexamethasone plasma levels than Tobradex®.

Based on these Results OCS-01 is Much Less Likely to Cause Systemic Side Effects Compared to What could be Expected with Other Eye Drop Formulations with Similar Dose.

### Example 2: Phase II/III Clinical Trial in Subject with Diabetic Macular Edema—Study DX219 DIAMOND

Study DX219 (DIAMOND) is a Phase 2/3 double-masked, randomized, 2-stage, multicenter study of the efficacy and safety of OCS-01 eye drop formulation in subjects with diabetic macular edema. There are two stages of this study. The patients enrolled in the Stage 1 portion of the study will not be participating in the Stage 2 of the study.

Stage 1 was conducted in 39 sites across the USA and Europe with 148 patients randomized 2:1 to receive OCS-01 (n=100) or vehicle (n=48) six times daily for a six-week induction phase and then three times daily for a subsequent six-week maintenance phase.

The key entry criteria for the study are:
Age 18-85 years
Type 1 or 2 DM.
BCVA (Best corrected visual acuity) of 65-24 ETDRS (Snellen equivalent 20/50-20/320)
Central Subfield Thickness (CST)>310 μm, as measured by SD-OCT
Previous treatment allowed (washout)

The endpoints for the Stage 1 of DX219 are:
Primary endpoint: Mean change in BCVA from baseline at 6 weeks
Mean change in BCVA from baseline at 12 weeks.
% of patients with a ≥3-line gain in BCVA at 6 and 12 weeks
Mean change in CST assessed by SD-OCT.
AUC of BCVA changes
% of patients with a ≥1-line or ≥2-line gain in BCVA at each post-baseline visit
Adverse events

Subjects were randomized at 2:1 ratio (OCS-01 vs. Vehicle) and the drug or vehicle were administered 6 times per day during the induction phase of 6 weeks and three times per day during the maintenance phase for another 6 weeks.

There were total of 148 patients randomized, 100 in OCS-01 group and 48 in vehicle group. Out of these patients, 85 in OCS-01 and 39 in vehicle group completed the 12-week study.

The study met the primary endpoint of LS mean change from baseline in BCVA letters core at week 6 with statistical significance, p=0.007 (95% CI, 1.1-7.0), versus (vs) vehicle (OCS-01:7.2 letters vs vehicle: 3.1 letters, p=0.007) demonstrating strong visual gain in the treatment arm. The effect was sustained to Week 12 with statistical significance (OCS-01:7.6 letters vs vehicle 3.7 letters, p=0.016). Furthermore, there was a higher percentage of patients in the OCS-01 group who achieved ≥15-letter improvement in BCVA from baseline vs vehicle at Week 6 (OCS-01:25.3% vs vehicle: 9.8%, p=0.015), which was sustained to Week 12 (OCS-01:27.4% vs vehicle 7.5%, p=0.009). (see Table 1). The LS mean change from baseline in BCVA at each visit is shown in FIG. 1.

TABLE 1
Study DX 219, Stage 1, Visual Acuity Results
Summary for ITT and Per Protocol Populations

| | | LS* mean change from baseline | | | | | |
|---|---|---|---|---|---|---|---|
| | | in BCVA letters | | | ≥15 letter gain | | |
| | | OCS-01 | Vehicle | P | OCS-01 | Vehicle | P |
| Week 6 | ITT | 7.2 | 3.1 | 0.007 | 25.3% | 9.8% | 0.015 |
| | Per | 7.4 | 3.4 | 0.015 | 24.7% | 10.3% | 0.033 |
| | protocol | | | | | | |
| Week 12 | ITT | 7.6 | 3.7 | 0.016 | 27.4% | 7.5% | 0.009 |
| | Per | 7.7 | 4.1 | 0.034 | 27.8% | 7.9% | 0.007 |
| | protocol | | | | | | |

ITT, intention to treat (OCS-01 n = 100, vehicle n = 48); per protocol (OCS-01 n = 89, vehicle n = 39)
*Least squares

The study also met the secondary endpoint of mean change from baseline in CST with robust statistical significance, the results in FIG. 2 show the CST change at each post-baseline visit.

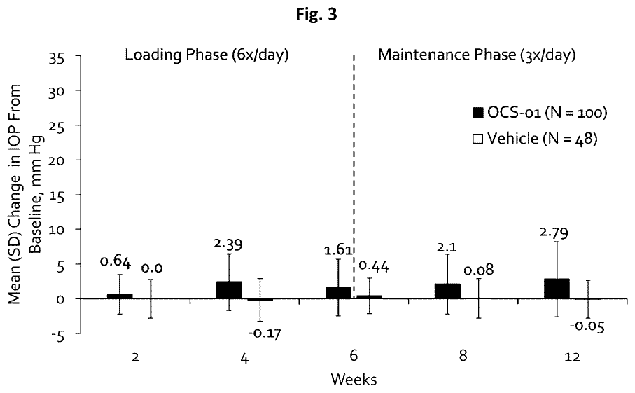

Most common adverse events were: diabetic macular edema, increase IOP, hypertension, ocular hypertension, and macular edema. IOP related events are shown in Table 2, and FIG. 3 shows the mean increase in IOP mmHg above baseline at time points throughout the study. Serious Treatment-Emergent Adverse Events (>2.0% in the OCS-01 Arm or >4.0% in the Vehicle Arm) are shown in Table 3.

TABLE 2
Study DX219, Stage 1, Intraocular
Pressure (IOP) related events

| | OCS-01 | | Vehicle | |
|---|---|---|---|---|
| | n = 100 | | n = 48 | |
| | n (%) | | n (%) | |
| Any IOP related Adverse | 22/100 | (22.0) | 1/48 | (2.1) |
| Event | | | | |
| ≥10 mm Hg IOP change | 16/97 | (16.5%) | 0/47 | (0) |
| from baseline at any visit | | | | |
| ≥25 mm Hg IOP at any visit | 19/97 | (19.6) | 1/47 | (2.1) |
| ≥35 mm Hg IOP at any visit | 1/97 | (1.0) | 0/47 | (o) |

| | | |
|---|---|---|
| IOP lowering medications | 11/22 | 1/1 |
| administered for Adverse | | |
| Events | | |

Safety population (OCS-01 n = 100, vehicle n = 48)

TABLE 3
Study DX219, Stage 1, Treatment-Emergent
Serious Adverse Events (SAE)

| | OCS-01 | Vehicle |
|---|---|---|
| | (n = 100) | (n = 48) |
| | n(%) | n(%) |
| Any ocular SAE | 1(1.0) | 0(0.0) |
| Vitreous hemorrhage | 1(1.0) | 0(0.0) |
| Any non-ocular SAE | 4(4.0) | 3(6.3) |
| Death | 1(1.0) | 0(0.0) |

One fatal adverse event occurred in the OCS-01 group. The event was determined unrelated to study drug.

Table 4 summarizes clinically-relevant ocular outcome data originating from the DX-219 Stage 1 study and OZURDEX clinical study for illustration purposes.

TABLE 4
Comparative data between DX219 and OZURDEX ®

| | OCS-01 | |
|---|---|---|
| | DX219 | Ozurdex¹ |
| Mean Change in BVCA Letters | 7.6 | 6.0 |
| 12 weeks | | |
| Gain of ≥15 Letters (3-line gain) | 27.4% | 12.5% |
| in BCVA 12 weeks | | |
| IOP elevation ≥10 mm Hg | 16.5% | 28.0% |
| from Baseline Any Visit | | |
| IOP elevation ≥25 mm Hg | 19.6% | 33% |
| from Baseline Any Visit | | |
| IOP elevation ≥35 mm Hg | 1.0% | 6% |
| from Baseline Any Visit | | |
| IOP lowering medication | 11% | 42% |

¹Ozurdex Prescribing Information, pooled data from two registrational studies.

### CONCLUSION

OCS-01, in a 3-month evaluation in humans established clinical efficacy endpoints, using a specific dosage regimen of 6 drops/day during 6 weeks and then 3 drops/day during 6 weeks (main BCVA change, proportion of patients with 3 lines gain).

OCS-01 was well-tolerated with no unexpected adverse events observed which is what is needed to encourage patient compliance to the treatment.

We claim:

1. A method of treating diabetic macular edema (DME) in a human subject in need thereof, comprising
(i) an induction phase of topically administering to an affected eye of the subject, an eye drop formulation comprising 1.5% dexamethasone, at a dosing of 5, 6, or 7 drops a day, followed by,
(ii) a maintenance phase of topically administering to the affected eye of the subject, the eye drop formulation at a dosing of 1, 2, or 3, drops a day;
wherein the eye drop formulation further comprises:
−12% to 16% of γ-cyclodextrin;
−2% to 2.7% of a poloxamer;
−0% to 0.1% of a chelating agent;
−0% to 1% of an electrolyte;
−0% to 0.6% of an antioxidant;
and wherein the pH of the eye drop formulation is between 4.7 and 6.0; and
the % are % by weight based on the volume of the eye drop formulation.

2. The method of claim 1, wherein the eye drop formulation is a microsuspension comprising solid complexes of dexamethasone and γ-cyclodextrin.

3. The method of claim 2, wherein the solid complexes are microparticles with a diameter D50 ranging from 1 μm to 20 μm.

4. The method of claim 3, wherein the antioxidant is sodium thiosulfate.

5. The method of claim 4, wherein eye drop formulation comprises sodium thiosulfate at a concentration of 0.1% to 0.4%.

6. The method of claim 5, wherein the eye drop formulation is in a plastic vial.

7. The method of claim 6, wherein the induction phase comprises topically administering the eye drop formulation at a dosing of 5, 6, or 7 drops a day for a period ranging from 5 days to up to 6 weeks.

8. The method of claim 7, wherein the induction phase comprises topically administering the eye drop formulation at a dosing of 6 drops a day.

9. The method of claim 8, wherein the maintenance phase comprises topically administering the eye drop formulation at a dosing of 1, 2, or 3 drops a day for a period of at least 3 weeks.

10. The method of claim 9, wherein the maintenance phase comprises topically administering the eye drop formulation at a dosing of 3 drops a day.

11. The method of claim 1, wherein the maintenance phase comprises topically administering the eye drop formulation at a dosing of 1, 2, or 3 drops a day for a period of at least 3 weeks.

12. The method of claim 11, wherein the maintenance phase comprises topically administering the eye drop formulation at a dosing of 3 drops a day.

13. The method of claim 1, wherein the induction phase comprises topically administering the eye drop formulation at a dosing of 5, 6, or 7 drops a day for a period ranging from 5 days to up to 6 weeks.

14. The method of claim 13, wherein the induction phase comprises topically administering the eye drop formulation at a dosing of 6 drops a day.

15. The method of claim 14, wherein the maintenance phase comprises topically administering the eye drop formulation at a dosing of 1, 2, or 3 drops a day for a period of at least 3 weeks.

16. The method of claim 15, wherein the maintenance phase comprises topically administering the eye drop formulation at a dosing of 3 drops a day.

17. The method of claim 1, wherein the subject has an inadequate response to a VEGF inhibitor treatment.

18. The method of claim 1, wherein the subject is VEGF inhibitor naïve, with retinal thickening in the affected eye due to diabetic macular edema.

19. The method of claim 1, wherein the mean ETDRS Best Corrected Visual Acuity is improved to at least 5 ETDRS letters at the end of the induction phase in the subject suffering from DME as compared to baseline.

20. The method of claim 1, wherein the mean ETDRS Best Corrected Visual Acuity is improved to at least 7 ETDRS letters at the end of the induction phase in the subject suffering from DME as compared to baseline.

21. The method of claim 1, wherein the central subfield thickness (CST) is reduced by at least 50 μm as measured from baseline, after 6 weeks of treatment in the subject suffering from DME.

22. The method of claim 1, wherein the eye drop formulation is preservative free.

23. The method of claim 1, wherein the chelating agent is 0.1% disodium edetate.

24. The method of claim 1, wherein the electrolyte is sodium chloride.

25. The method of claim 1, wherein the antioxidant is sodium thiosulfate.

26. The method of claim 25, wherein the eye drop formulation comprises sodium thiosulfate at a concentration of 0.1% to 0.4%.

27. The method of claim 25, wherein the eye drop formulation comprises sodium thiosulfate at a concentration of about 0.3%.

28. The method of claim 1, wherein the eye drop formulation is a microsuspension comprising solid complexes with a diameter D50 ranging from 1 μm to 10 μm.
